SEQUENCE LISTING (32 sequences)

SEQ ID NO: 1 - DNA, 4053 bp, Homo sapiens
  CDS (135)..(3710)
caccacagtaggtccctcggctcagtcggcccagcccctctcagtcctccccaacccccacaaccgcccgcggctctgagacgcggccccggcggcggcggcagcagctgcagcatcatctccaccctccagccatggaagacctggaccagtctcctctggtctcgtcctcggacagcccaccccggccgcagcccgcgttcaagtaccagttcgtgagggagcccgaggacgaggaggaagaagaggaggaggaagaggaggacgaggacgaagacctggaggagctggaggtgctggagaggaagcccgccgccgggctgtccgcggccccagtgcccaccgcccctgccgccggcgcgcccctgatggacttcggaaatgacttcgtgccgccggcgccccggggacccctgccggccgctccccccgtcgccccggagcggcagccgtcttgggacccgagcccggtgtcgtcgaccgtgcccgcgccatccccgctgtctgctgccgcagtctcgccctccaagctccctgaggacgacgagcctccggcccggcctccccctcctcccccggccagcgtgagcccccaggcagagcccgtgtggaccccgccagccccggctcccgccgcgcccccctccaccccggccgcgcccaagcgcaggggctcctcgggctcagtggatgagaccctttttgctcttcctgctgcatctgagcctgtgatacgctcctctgcagaaaatatggacttgaaggagcagccaggtaacactatttcggctggtcaagaggatttcccatctgtcctgcttgaaactgctgcttctcttccttctctgtctcctctctcagccgcttctttcaaagaacatgaataccttggtaatttgtcaacagtattacccactgaaggaacacttcaagaaaatgtcagtgaagcttctaaagaggtctcagagaaggcaaaaactctactcatagatagagatttaacagagttttcagaattagaatactcagaaatgggatcatcgttcagtgtctctccaaaagcagaatctgccgtaatagtagcaaatcctagggaagaaataatcgtgaaaaataaagatgaagaagagaagttagttagtaataacatccttcataatcaacaagagttacctacagctcttactaaattggttaaagaggatgaagttgtgtcttcagaaaaagcaaaagacagttttaatgaaaagagagttgcagtggaagctcctatgagggaggaatatgcagacttcaaaccatttgagcgagtatgggaagtgaaagatagtaaggaagatagtgatatgttggctgctggaggtaaaatcgagagcaacttggaaagtaaagtggataaaaaatgttttgcagatagccttgagcaaactaatcacgaaaaagatagtgagagtagtaatgatgatacttctttccccagtacgccagaaggtataaaggatcgttcaggagcatatatcacatgtgctccctttaacccagcagcaactgagagcattgcaacaaacatttttcctttgttaggagatcctacttcagaaaataagaccgatgaaaaaaaaatagaagaaaagaaggcccaaatagtaacagagaagaatactagcaccaaaacatcaaacccttttcttgtagcagcacaggattctgagacagattatgtcacaacagataatttaacaaaggtgactgaggaagtcgtggcaaacatgcctgaaggcctgactccagatttagtacaggaagcatgtgaaagtgaattgaatgaagttactggtacaaagattgcttatgaaacaaaaatggacttggttcaaacatcagaagttatgcaagagtcactctatcctgcagcacagctttgcccatcatttgaagagtcagaagctactccttcaccagttttgcctgacattgttatggaagcaccattgaattctgcagttcctagtgctggtgcttccgtgatacagcccagctcatcaccattagaagcttcttcagttaattatgaaagcataaaacatgagcctgaaaaccccccaccatatgaagaggccatgagtgtatcactaaaaaaagtatcaggaataaaggaagaaattaaagagcctgaaaatattaatgcagctcttcaagaaacagaagctccttatatatctattgcatgtgatttaattaaagaaacaaagctttctgctgaaccagctccggatttctctgattattcagaaatggcaaaagttgaacagccagtgcctgatcattctgagctagttgaagattcctcacctgattctgaaccagttgacttatttagtgatgattcaatacctgacgttccacaaaaacaagatgaaactgtgatgcttgtgaaagaaagtctcactgagacttcatttgagtcaatgatagaatatgaaaataaggaaaaactcagtgctttgccacctgagggaggaaagccatatttggaatcttttaagctcagtttagataacacaaaagataccctgttacctgatgaagtttcaacattgagcaaaaaggagaaaattcctttgcagatggaggagctcagtactgcagtttattcaaatgatgacttatttatttctaaggaagcacagataagagaaactgaaacgttttcagattcatctccaattgaaattatagatgagttccctacattgatcagttctaaaactgattcattttctaaattagccagggaatatactgacctagaagtatcccacaaaagtgaaattgctaatgccccggatggagctgggtcattgccttgcacagaattgccccatgacctttctttgaagaacatacaacccaaagttgaagagaaaatcagtttctcagatgacttttctaaaaatgggtctgctacatcaaaggtgctcttattgcctccagatgtttctgctttggccactcaagcagagatagagagcatagttaaacccaaagttcttgtgaaagaagctgagaaaaaacttccttccgatacagaaaaagaggacagatcaccatctgctatattttcagcagagctgagtaaaacttcagttgttgacctcctgtactggagagacattaagaagactggagtggtgtttggtgccagcctattcctgctgctttcattgacagtattcagcattgtgagcgtaacagcctacattgccttggccctgctctctgtgaccatcagctttaggatatacaagggtgtgatccaagctatccagaaatcagatgaaggccacccattcagggcatatctggaatctgaagttgctatatctgaggagttggttcagaagtacagtaattctgctcttggtcatgtgaactgcacgataaaggaactcaggcgcctcttcttagttgatgatttagttgattctctgaagtttgcagtgttgatgtgggtatttacctatgttggtgccttgtttaatggtctgacactactgattttggctctcatttcactcttcagtgttcctgttatttatgaacggcatcaggcacagatagatcattatctaggacttgcaaataagaatgttaaagatgctatggctaaaatccaagcaaaaatccctggattgaagcgcaaagctgaatgaaaacgcccaaaataattagtaggagttcatctttaaaggggatattcatttgattatacgggggagggtcagggaagaacgaaccttgacgttgcagtgcagtttcacagatcgttgttagatctttatttttagccatgcactgttgtgaggaaaaattacctgtcttgactgccatgtgttcatcatcttaagtattgtaagctgctatgtatggatttaaaccgtaatcatatctttttcctatctgaggcactggtggaataaaaaacctgtatattttactttgttgcagatagtcttgccgcatcttggcaagttgcagagatggtggagctag

SEQ ID NO: 2 - protein, 1192 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 3 - DNA, 1441 bp, Homo sapiens
ccaacccctacgatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctgacccccag

SEQ ID NO: 4 - protein, 473 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 5 - protein, 57 aa, Homo sapiens
IFSAELSKTSVVDLLYWRDIKKTGGRIYKGVIQAIQKSDEGHPFRAYLESEVAISEE

SEQ ID NO: 6 - protein, 471 aa, Rattus sp.
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKASVLEPGRPASVGNALKGRVPPGDTPPGNGSGPRHINDSPFGTLPGSAEPPLTALRPGGSEPPGLPTTGPRRRPGCSRKNRTRSHCRLGQAGSGSSGTGDAEGSGALPALACSLAPLGLALVLWTVLGPC

SEQ ID NO: 7 - protein, 15 aa, Artificial Sequence
  Chemically synthesized linker sequence that separates two coding regions in a fusion protein
GGGGSGGGGSGGGGS

SEQ ID NO: 8 - protein, 15 aa, Artificial Sequence
  Chemically synthesized linker sequence that separates two coding regions in a fusion protein
ESGRSGGGGSGGGGS

SEQ ID NO: 9 - protein, 14 aa, Artificial Sequence
  Chemically synthesized linker sequence that separates two coding regions in a fusion protein
EGKSSGSGSESKST

SEQ ID NO: 10 - protein, 15 aa, Artificial Sequence
  Chemically synthesized linker sequence that separates two coding regions in a fusion protein
EGKSSGSGSESKSTQ

SEQ ID NO: 11 - protein, 14 aa, Artificial Sequence
  Chemically synthesized linker sequence that separates two coding regions in a fusion protein
EGKSSGSGSESKVD

SEQ ID NO: 12 - protein, 14 aa, Artificial Sequence
  Chemically synthesized linker sequence that separates two coding regions in a fusion protein
GSTSGSGKSSEGKG

SEQ ID NO: 13 - protein, 18 aa, Artificial Sequence
  Chemically synthesized linker sequence that separates two coding regions in a fusion protein
KESGSVSSEQLAQFRSLD

SEQ ID NO: 14 - protein, 16 aa, Artificial Sequence
  Chemically synthesized linker sequence that separates two coding regions in a fusion protein
ESGSVSSEELAFRSLD

SEQ ID NO: 15 - protein, 8 aa, Artificial Sequence
  Chemically synthesized FLAG peptide
DYKDDDDK

SEQ ID NO: 16 - protein, 8 aa, Artificial Sequence
  Chemically synthesized FLAG peptide
DYKDEDDK

SEQ ID NO: 17 - protein, 9 aa, Artificial Sequence
  Chemically synthesized Strep epitope
AWRHPQFGG

SEQ ID NO: 18 - protein, 11 aa, Artificial Sequence
  Chemically synthesized VSV-G epitope
YTDIEMNRLGK

SEQ ID NO: 19 - protein, 6 aa, Artificial Sequence
  Chemically synthesized epitope with a poly-His sequence
HHHHHH

SEQ ID NO: 20 - protein, 13 aa, Influenza virus
YPYDVPDYAIEGR

SEQ ID NO: 21 - protein, 11 aa, Homo sapiens
EQKLLSEEDLN

SEQ ID NO: 22 - protein, 24 aa, Homo sapiens
IFSAELSKTSVVDLLYWRDIKKTG

SEQ ID NO: 23 - protein, 21 aa, Homo sapiens
IFSAELSKTSVVDLLYWRDIK

SEQ ID NO: 24 - protein, 20 aa, Homo sapiens
IFSAELSKTSVVDLLYWRDI

SEQ ID NO: 25 - protein, 19 aa, Homo sapiens
IFSAELSKTSVVDLLYWRD

SEQ ID NO: 26 - protein, 23 aa, Homo sapiens
FSAELSKTSVVDLLYWRDIKKTG

SEQ ID NO: 27 - protein, 19 aa, Homo sapiens
LSKTSVVDLLYWRDIKKTG

SEQ ID NO: 28 - protein, 14 aa, Homo sapiens
VVDLLYWRDIKKTG

SEQ ID NO: 29 - protein, 10 aa, Homo sapiens
IFSAELSKTS

SEQ ID NO: 30 - protein, 25 aa, Homo sapiens
MDGQKKNWKDKVVDLLYWRDIKKTG

SEQ ID NO: 31 - protein, 24 aa, Artificial Sequence
  Biotin labeled Ng24
IFSAELSKTSVVDLLYWRDIKKTG

SEQ ID NO: 32 - protein, 57 aa, Artificial Sequence; identical to SEQ ID NO: 5
  Biotin labeled 24/32

(19) United States
(12) Patent Application Publication
(10) Pub. No.: US 2009/0111753 A1
(43) Pub. Date: Apr. 30, 2009

(54) Nogo-A Polypeptide Fragments, Variant Nogo Receptor-1 Polypeptides, and Uses Thereof

(75) Inventor: Stephen M. Strittmatter, Guilford, CT (US)

Correspondence Address: STERNE, KESSLER, GOLDSTEIN & FOX, P.L.L.C., 1100 NEW YORK AVE., N.W., WASHINGTON, DC, 20005, US

(73) Assignee: Yale University, New Haven, CT (US)

(21) Appl. No.: 11/576,413

(22) PCT Filed: Oct. 3, 2005
(86) PCT No.: PCT/US05/35719

§ 371 Date: Feb. 19, 2008

Related U.S. Application Data
(60) Provisional application No. 60/615,371, filed on Oct. 1, 2004.

(51) Int. Cl. A61K 38/10; C07K 7/00; C07K 14/00; C12N 5/10

(52) U.S. Cl. 514/13; 530/326; 530/325; 530/324; 435/325

(57) ABSTRACT

Nogo, MAG, and OMgp are myelin-derived proteins that bind to a neuronal Nogo-66 Receptor (NgR) to limit axonal regeneration after CNS injury. Nogo-A protein may play the most prominent role in vivo, perhaps because its action is mediated both by NgR and by other receptors. Here, we extend our previous analysis of Nogo-A and NgR functional domains. In addition to a NgR-dependent Nogo-66 inhibitory domain and a NgR-independent Amino-Nogo-A specific domain, we identify a third Nogo-A specific domain that binds to NgR with nanomolar affinity. This third domain of 19 amino acids (aa) does not alter cell spreading or axonal outgrowth. Ala-scanning mutagenesis of surface residues in NgR partially distinguishes ligand binding sites for the two Nogo domains and for MAG, OMgp and Lingo-1. Fusion of the two NgR-binding Nogo-A domains creates a ligand with ten-fold enhanced affinity for NgR and converts a NgR antagonist peptide to an agonist. Thus, inhibition of axonal regeneration by NgR occurs after binding a subnanomolar bipartite Nogo-A ligand at a site partly overlapping with that for MAG and OMgp.

### BACKGROUND OF THE INVENTION

1. Field of the Invention

This invention relates to neurobiology, neurology and pharmacology. More particularly, the invention relates to neurons and compositions and methods for mediating axonal growth.

2. Background Art

In the brain and spinal cord of adult mammals, axonal connections are static. If connections are severed by injury, little or no regrowth of axons occurs. Extrinsic to the neuron, astroglial scars and CNS myelin inhibit axonal growth (Horner, P. J. and Gage, F. H., Nature 407:963-970 (2000); McGee, A. W. and Strittmatter, S. M., Trends Neurosci. 26:193-198 (2003)). If the environment surrounding the adult CNS axon is altered, then axonal growth can occur (Benfey, M. and Aguayo, A. J., Nature 296:150-152 (1982); David, S, and Aguayo, A. J., Science 214:931-933 (1981); Richardson, P. M., et al., Nature 284:264-265 (1980)). From CNS myelin, three proteins capable of inhibiting axonal growth in vitro have been isolated, Nogo, MAG and OMgp McGee, A. W. and Strittmatter, S. M., Trends Neurosci. 26:193-198 (2003)).

Nogo exists in three isoforms, all of which share a carboxyl terminal segment that contains two hydrophobic segments (Chen, M. S., et al., Nature 403:434-439 (2000); GrandPre, T., et al., Nature 403:439-444 (2000); McGee, A. W. and Strittmatter, S. M., Trends Neurosci. 26:193-198 (2003); Prinjha, R., et al., Nature 403:383-384 (2000)). The three isoforms have distinct hydrophilic amino terminal segments and Nogo-A is the primary form produced by oligodendrocytes in CNS myelin (Chen, M. S., et al., Nature 403:434-439 (2000); GrandPre, T., et al., Nature 403:439-444 (2000); Huber, A. B., et al., J. Neurosci. 22:3553-3567 (2002); Wang, X., et al., J. Neurosci. 22:5505-5515 (2002c)). Nogo-A has been shown to possess two inhibitory domains. The inhibitory Nogo-66 domain in the carboxyl region is flanked by the two hydrophobic segments and is detectable on the surface of oligodendrocytes (Fournier, A. E., et al., Nature 409:341-346 (2001); GrandPre, T., et al., Nature 403:439-444 (2000); Oertle, T., et al., J. Neurosci. 23:5393-5406 (2003b)). The amino terminal segment of Nogo-A independently exhibits axon inhibition (Chen, M. S., et al., Nature 403:434-439 (2000); Fournier, A. E., et al., Nature 409:341-346 (2001)); a central Δ20 region appears most critical for this activity (Oertle, T., et al., J. Neurosci. 23:5393-5406 (2003b)). The Amino-Nogo domain, like the Nogo-66 domain, has been detected on the surface of oligodendrocytes and two conformations for Nogo-A have been proposed (Chen, M. S., et al., Nature 403:434-439 (2000); GrandPre, T., et al., Nature 403:439-444 (2000); (Oertle, T., et al., J. Neurosci. 23:5393-5406 (2003b)). In one, the amino and carboxyl terminus are cytosolic and the Nogo-66 loop is extracellular with two transmembrane segments. In an alternate topology, the first hydrophobic segment loops into and out of the plasma membrane without forming a transmembrane segment, so that the Amino-Nogo and Nogo-66 are located on the same side of the lipid bilayer.

Antibody or peptide perturbation of the Nogo pathway leads to an enhanced axonal growth, plasticity and functional recovery after spinal injury or stroke (Bregman, B. S., et al., Nature 378:498-501 (1995); GrandPre, T., et al., Nature 417:547-551 (2002); Lee, J. K., et al., J. Neurosci. 24:6209-6217 (2004); Li, S, and Strittmatter, S. M., J. Neurosci. 23:4219-4227 (2003); Schnell, L. and Schwab, M. E., Nature 343:269-272 (1990); Wiessner, C., et al., J. Cereb. Blood Flow Metab. 23:154-165 (2003)). Genetic studies of Nogo function have provided conflicting data on the essential role for Nogo in axonal regeneration (Kim, J. E., et al., Neuron 38:187-199 (2003b); Simonen, M., et al., Neuron, 38:201-211 (2003); Zheng, B., et al., Neuron. 38:213-224 (2003)). While Nogo-A-I-myelin has reduced inhibitory activity in all studies, in two studies this was associated with a degree of axonal regeneration in vivo and in another study with no regeneration in vivo (Kim, J. E., et al., Neuron 38:187-199 (2003b); Simonen, M., et al., Neuron, 38:201-211 (2003); Zheng, B., et al., Neuron. 38:213-224 (2003)). Transgenic expression of Nogo in the periphery is sufficient to slow otherwise rapid regeneration (Kim, J. E., et al., Mol. Cell. Neurosci. 23:451-459 (2003a); Pot, C., et al., J. Cell Biol. 159:29-35 (2002)). Mice lacking MAG have been reported to lack CNS axonal regeneration (Bartsch, U., et al., Neuron 15:1375-1381 (1995)), although peripheral regeneration may be enhanced in certain genetic backgrounds (Schafer, M., et al., Neuron 16:1107-1113 (1996)).

A receptor for the Nogo-66 domain was identified by expression cloning (Nogo-66 Receptor, NgR) (Fournier, A. E., et al., Nature 409:341-346 (2001)). This protein is expressed selectively in postnatal neurons and mediates responsiveness to Nogo-66. NgR is a leucine-rich repeat (LRR) containing protein that is GPI-anchored to the surface of the neurons. The LRR domain forms the ligand binding site and its structure has been determined (Barton, W. A., et al., Embo J. 22:3291-3302 (2003); Fournier, A. E., et al., J. Neurosci. 22:8876-8883 (2002); He, X. L., et al., Neuron 38:177-185 (2003)). Remarkably, MAG and OMgp bind to the LRR domain of the same NgR protein to inhibit axonal growth in vitro (Domeniconi, M., et al., Neuron 35:283-290 (2002); Liu, B. P., et al., Science 297:1190-1193 (2002); Wang, K. C., et al., Nature 417:941-944 (2002b)). In vivo, genetic deletion of NgR allows some axonal fibers to sprout and enhances functional recovery after spinal cord transection (Kim, J. E., et al., Neuron 44:439-451 (2004)). Co-receptors are required to transmit a signal from NgR to the cell interior to regulate axonal motility. Both the p75^{NTR} and Lingo-1 transmembrane proteins have been implicated in NgR signal transduction (Mi, S., et al., Nat. Neurosci. 7:221-228 (2004); Wang, K. C., et al., Nature 420:74-78 (2002a); Wong, S. T., et al., Nat. Neurosci. 5:1302-1308 (2002)). However, neither receptors for the Amino-Nogo domain nor the molecular basis of NgR interaction with multiple ligands have been defined.

Our initial functional analysis of Nogo-A activity had separated the Amino-Nogo domain from the Nogo-66 domain (Fournier, A. E., et al., Nature 409:341-346 (2001)). We had demonstrated that NgR is a receptor for Nogo-66, but that Amino-Nogo utilizes other mechanisms. Here, we have uncovered an additional activity not revealed in morphologic assays.

### BRIEF SUMMARY OF THE INVENTION

The present invention is based on the discovery that the Amino-Nogo domain of Nogo-A harbors a region that interacts with a central binding domain in the NgR. The combination of Nogo-66 with this Amino-Nogo domain creates a substantially higher affinity NgR ligand, which is likely to be of central importance in limiting axonal regeneration in vivo. Furthermore, the NgR utilizes certain residues to interact with multiple ligands in the central binding domain and certain surrounding residues to interact with specific ligands. Based on these discoveries, the invention relates to molecules and methods useful for enhancing axonal growth inhibition in CNS neurons.

In some embodiments, the invention provides an isolated polypeptide fragment of 30 residues or less, comprising an amino acid sequence that is at least 90% identical to a reference amino acid sequence selected from the group consisting of: (a) amino acids 995 to 1013 of SEQ ID NO:2; (b) amino acids 995 to 1014 of SEQ ID NO:2; (c) amino acids 995 to 1015 of SEQ ID NO:2; (d) amino acids 995 to 1016 of SEQ ID NO:2; (e) amino acids 995 to 1017 of SEQ ID NO:2; (f) amino acids 995 to 1018 of SEQ ID NO:2; (g) amino acids 992 to 1018 of SEQ ID NO:2; (h) amino acids 993 to 1018 of SEQ ID NO:2; and (i) amino acids 994 to 1018 of SEQ ID NO:2 and where the polypeptide binds NgR1. In some embodiments, the invention provides that the polypeptide fragment of the invention is at least 95% identical to the reference amino acid sequence. In other embodiments, the polypeptide fragment is identical to the reference amino acid sequence.

In some embodiments, the invention provides an isolated polypeptide fragment of 200 residues or less comprising a first amino acid sequence that is at least 90% identical to amino acids 995 to 1018 of SEQ ID NO:2 and where the first amino acid sequence is linked to amino acids 1055 to 1086 of SEQ ID NO:2 and where the polypeptide fragment binds NgR1. In some embodiments, the first amino acid sequence comprises amino acids 995 to 1018 of SEQ ID NO:2 linked to amino acids 1055 to 1086 of SEQ ID NO:2. In other embodiments, the first amino acid sequence comprises amino acids 950 to 1018 of SEQ ID NO:2 linked to amino acids 1055 to 1086 of SEQ ID NO:2. In some embodiments, the polypeptide fragment of the invention enhances NgR-mediated neurite outgrowth inhibition. In some embodiments, the polypeptide fragment comprises and/or consists essentially of SEQ ID NO:5.

In some embodiments, the invention provides a polypeptide of the invention that is modified. In some embodiments, the modification is biotinylation.

In some embodiments the invention further provides that the polypeptide is fused to a heterologous polypeptide. In some embodiments the heterologous polypeptide is Glutathione S-transferase (GST). In some embodiments the heterologous polypeptide is histidine tag (His tag). In some embodiments the heterologous polypeptide is alkaline phosphatase (AP). In some embodiments the heterologous polypeptide is Fc.

In some embodiments the invention provides an isolated human NgR1 polypeptide comprising amino acids 27 to 473 of SEQ ID NO:4, except for amino acid substitution at least the amino acid positions selected from the group consisting of: (a) amino acids 67, 68 and 71; (b) amino acids 111, 113 and 114; (c) amino acids 133 and 136; (d) amino acids 158, 160, 182, and 186; (e) amino acid 163; and (f) amino acids 232 and 234; where the NGR1 polypeptide does not bind to any of Nogo 66, OMgp, Mag or Lingo-1. In other embodiments, the invention provides an isolated human NgR1 polypeptide comprising amino acids 27 to 473 of SEQ ID NO:4, except for amino acid substitutions at least the amino acid positions selected from the group consisting of: (a) amino acids 78 and 81; (b) amino acids 87 and 89; (c) amino acids 89 and 90; (d) amino acids 95 and 97; (e) amino acid 108; (f) amino acids 117, 119 and 120; (g) amino acid 139; (h) amino acid 210; and (i) amino acids 256 and 259; where the NgR polypeptide selectively binds to at least one but not all of Nogo 66, OMgp, Mag or Lingo-1.

Additional embodiments that are envisioned include a polynucleotide expressing the polypeptide or fragment thereof of the present invention, vectors comprising the polynucleotides, and host cells comprising the polynucleotides and expressing the polypeptides of the invention.

Additional embodiments of the invention include compositions comprising the polypeptides, polynucleotides, vectors or host cells of the invention and in certain embodiments a pharmaceutically acceptable carrier. The composition can be formulated for administration by a route selected from the group consisting of parenteral administration, subcutaneous administration, intravenous administration, intramuscular administration, intraperitoneal administration, transdermal administration, buccal administration, oral administration and microinfusion administration. The composition can further comprise a carrier.

### BRIEF DESCRIPTION OF THE DRAWINGS/FIGURES

FIG. 1A. Binding of Amino-Nogo fragments to NgR. Schematic drawing of Amino-Nogo fragments A, B and Δ20.

FIG. 1B. Binding of alkaline phosphatase (AP) fused Amino-Nogo fragment B (AmNg B), but not fragments A (AmNg A) or Δ20 to COS-7 cells expressing NgR. Conditioned media from HEK293T cells containing AP fusion protein of indicated concentrations were applied to untransfected or COS-7 cells expressing NgR and bound AP was stained.

FIG. 1C. Amino-Nogo-A-24 is the binding domain for NgR in Amino-Nogo. Different fragments of Amino Nogo as indicated were fused to AP and their binding to NgR was determined in cell binding assay as in (B).

FIG. 1D. Top: AP-Amino-Nogo-A-24 binding to NgR expressing COS-7 cells measured as a function of AP-Amino-Nogo-A-24 concentration. Bottom: Replotted data from top panel. Binding Kd was determined from four independent measurements.

FIG. 1E. Binding of AP fused Amino-Nogo fragments to dissociated E13 chick DRG neurons. Conditioned media from HEK293T cells containing AP fusion protein as indicated were applied to dissociated E13 chick DRG neurons and bound AP was stained.

FIG. 2A. Effects of Amino-Nogo fragments on fibroblast spreading and neurite outgrowth. Different effects of Amino-Nogo fragments on fibroblast spreading. COS-7 cells were allowed to attach and spread on slides with spots coated with 50 ng of dried GST fusion protein as indicated and stained for F-actin. GST-A: fusion protein of GST and A fragment (FIG. 1) of Amino-Nogo. GST-A, GST-B, GST-Δ20, GST-B4 and GST-B4C: fusion protein of GST and A, B, Δ20, B4 or B4C fragment (FIG. 1) of Amino-Nogo, respectively.

FIG. 2B. COS-7 cell area for experiments as in (A) was measured and plotted.

FIG. 2C. COS-7 cells were allowed to attach and spread on 96 well dishes coated with dried GST fusion proteins as indicated. Number of attached cells were counted and plotted as a function of the amount of various proteins dried per well in a 96 well dish.

FIG. 2D. Differential effects of Amino-Nogo fragments on neurite outgrowth. Dissociated neurons from E13 chick DRGs were plated on 96 well dishes coated with lpmol protein per well and stained for neurofilament localization.

FIG. 2E. Neurite length per neuron were measured and plotted as percentage of PBS control with increasing concentration of dried protein for experiment described in (E).

FIG. 3A. Binding of Amino-Nogo to NgR requires LRR repeats. Binding of AP or AP fused Nogo fragments to COS-7 cells expressing NgR mutants as indicated. AP-B and AP-B4: AP fusion protein of B or B4 fragment of Amino-Nogo. Surface expression of NgR mutants was detected using anti-Myc antibodies.

FIG. 3B. Amino-Nogo does not bind to NgR2 or NgR3. Conditioned media from transfected HEK293T cells containing 20 nM of indicated AP fusion protein were applied to COS-7 cells expressing mouse NgR1, human NgR2 or mouse NgR3 and bound AP was stained. Surface expression of NgR5 was detected using anti-Myc or anti-His antibodies. AP-AmNgA: AP fusion protein of Amino-Nogo fragment A. AP-AmNgB: AP fusion protein of Amino-Nogo fragment B.

FIG. 4A. Examples of N2R mutants that show differential binding to MgR ligands. Binding of AP or AP fused NgR ligands to COS-7 cells expressing different NgR mutants as indicated. The concentrations of ligands applied were: AP, 30 nM; AP-Ng66, 5 nM; AP-Ng33, 10 nM; AP-B4C, 10 nM; AP-B4C66, 0.5 nM; AP-Lingo-1, 10 nM; AP-OMGP, 10 nM; AP-MAG, 30 nM. These concentrations are close to the binding Kd of these proteins to NgR so that any decrease in Kd is reflected linearly in staining.

FIG. 4B. AP binding of NgR ligands to NgR mutants expressed as percentage of wild type NgR. AP after incubation with AP fused ligands, AP bound to COS-7 cells expressing NgR or NgR mutants were stained and measured.

FIG. 4C. Whole cell lysate of COS-7 cells expressing NgR mutants were subjected to SDS-PAGE and blotted with anti-NgR antibodies.

FIG. 5. Ligand binding sites in NgR. The molecular surface of NgR is illustrated with those residues essential for binding of all ligands labeled red, residues not required for ligand binding labeled blue and residues required for some ligands but not others labeled yellow. Residues required for Ng66 binding but not for B4C were indicated with arrows. This illustration was made using SwissPdbViewer software.

FIG. 6A. Fusion of B4C with Nogo66 creates a high affinity ligand for NgR. AP-B4C66 binding to NgR expressing COS-7 cells measured as a function of AP-B4C66 concentration.

FIG. 6B.: Replotted data from (A). Binding Kd was determined from four independent measurements.

FIG. 6C. B24/32 peptide inhibits neurite outgrowth. Dissociated neurons from E13 chick DRG were plated onto 96 well dishes coated with 500 pmol of dried peptides as indicated and stained for neurofilament localization.

FIG. 6D. Neurite length per neuron was measured and plotted as percentage of PBS control for experiments as in (C).

FIG. 7. Model for NgR signaling. NgR is the common receptor for oligodendrocyte proteins Nogo, MAG and OMgp. Ng19 region in Amino-Nogo and Nogo 66 bind to the LRR domain of NgR. Binding of Amino-Nogo-19 to NgR does not signal to inhibit outgrowth but the presence of both Amino-Nogo-19 and Ng66 in Nogo makes Nogo a high affinity agonist for NgR. MAG and OMgp also bind to the LRR domain of NgR. Δ20 region of Amino-Nogo does not bind to NgR but inhibits fibroblast spreading and neurite outgrowth, probably through an unidentified receptor present in multiple cell types. The amino terminal domain of Nogo shared by Nogo-A and Nogo-B might act through another unidentified receptor to regulate vascular remodeling.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

In order to further define this invention, the following terms and definitions are provided.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

Throughout this specification and claims, the word “comprise,” or variations such as “comprises” or “comprising,” indicate the inclusion of any recited integer or group of integers but not the exclusion of any other integer or group of integers.

As used herein, the term “consists of,” or variations such as “consist of” or “consisting of,” as used throughout the specification and claims, indicate the inclusion of any recited integer or group of integers, but that no additional integer or group of integers may be added to the specified method, structure or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

In the present invention, a “polypeptide fragment” refers to a short amino acid sequence of a larger polypeptide. Protein fragments may be “free-standing,” or comprised within a larger polypeptide of which the fragment forms a part of region. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids or more in length.

The terms “fragment,” “variant,” “derivative” and “analog” when referring to a polypeptide of the present invention include any polypeptide which retains at least some biological activity. Polypeptides as described herein may include fragment, variant, or derivative molecules therein without limitation, so long as the polypeptide still serves its function. Polypeptides or fragments thereof of the present invention may include proteolytic fragments, deletion fragments and in particular, fragments which more easily reach the site of action when delivered to an animal. Polypeptide fragments further include any portion of the polypeptide which comprises an antigenic or immunogenic epitope of the native polypeptide, including linear as well as three-dimensional epitopes. Polypeptides or fragments thereof of the present invention may comprise variant regions, including fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. Polypeptides or fragments thereof of the present invention may comprise conservative or non-conservative amino acid substitutions, deletions or additions. Polypeptides or fragments thereof of the present invention may also include derivative molecules. Variant polypeptides may also be referred to herein as “polypeptide analogs.” As used herein a “derivative” of a polypeptide or a polypeptide fragment refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

As used herein the term “disulfide bond” includes the covalent bond formed between two sulfur atoms. The amino acid cysteine comprises a thiol group that can form a disulfide bond or bridge with a second thiol group.

As used herein, “fusion protein” means a protein comprising a first polypeptide linearly connected, via peptide bonds, to a second, polypeptide. The first polypeptide and the second polypeptide may be identical or different, and they may be directly connected, or connected via a peptide linker (see below).

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can contain the nucleotide sequence of the full length cDNA sequence, including the untranslated 5′ and 3′ sequences, the coding sequences. A polynucleotide may comprise a conventional phosphodiester bond or a non-conventional bond (e.g., an amide bond, such as found in peptide nucleic acids (PNA)). The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. As used herein, polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide or fragment thereof of the present invention contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding a polypeptide or fragment thereof of the present invention. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the term “linked” refers to the joining together of two more elements or components, by whatever means including chemical conjugation or recombinant means. The term “linked” may mean directly fused by a peptide bond, indirectly fused with a spacer, as well as hooked together by means other than a peptide bond, e.g., through disulfide bonds or a non-peptide moiety.

A “linker” sequence is a series of one or more amino acids separating two polypeptide coding regions in a fusion protein. A typical linker comprises at least 5 amino acids. Additional linkers comprise at least 10 or at least 15 amino acids. In certain embodiments, the amino acids of a peptide linker are selected so that the linker is hydrophilic. The linker (Gly-Gly-Gly-Gly-Ser)₃ (SEQ ID NO:______) is a preferred linker that is widely applicable to many antibodies as it provides sufficient flexibility. Other linkers include Glu Ser Gly Arg Ser Gly Gly Gly Gly Ser Gly Gly Gly Gly Ser (SEQ ID NO:______), Glu Gly Lys Ser Ser Gly Ser Gly Ser Glu Ser Lys Ser Tlr (SEQ ID NO:______), Glu Gly Lys Ser Ser Gly Ser Gly Ser Glu Ser Lys Ser Thr Gln (SEQ ID NO:______), Glu Gly Lys Ser Ser Gly Ser Gly Ser Glu Ser Lys Val Asp (SEQ ID NO:______), Gly Ser Thr Ser Gly Ser Gly Lys Ser Ser Glu Gly Lys Gly (SEQ ID NO:______), Lys Glu Ser Gly Ser Val Ser Ser Glu Gln Leu Ala Gln Phe Arg Ser Leu Asp (SEQ ID NO:______), and Glu Ser Gly Ser Val Ser Ser Glu Glu Leu Ala Phe Arg Ser Leu Asp (SEQ ID NO:______). Examples of shorter linkers include fragments of the above linkers, and examples of longer linkers include combinations of the linkers above, combinations of fragments of the linkers above, and combinations of the linkers above with fragments of the linkers above.

As used herein, the terms “fused” or “fusion” with regard to polypeptides or polypeptide fragments are used interchangeably. These terms refer to the joining of two elements, either directly or indirectly, e.g., a peptide spacer, by a peptide bond. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (sbRNA), small interfering RNA (siRNA) or any other RNA product, and the translation of such mRNA into polypeptide(s), as well as any processes which regulate either transcription or translation. If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, phrases such as “a subject that would benefit from administration of a Nogo polypeptide or polypeptide fragment of the present invention” and “an animal in need of treatment” includes subjects, such as mammalian subjects, that would benefit from administration of a Nogo polypeptide or polypeptide fragment used, e.g., for detection (e.g., for a diagnostic procedure) and/or for treatment, i.e., palliation or prevention of a disease such as schizophrenia with a Nogo polypeptide or polypeptide fragment of the present invention. As described in more detail herein, the polypeptide or polypeptide fragment can be used in unconjugated form or can be conjugated, e.g., to a drug, prodrug, or an isotope.

As used herein, a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure”.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The invention is directed to certain Nogo polypeptides and polypeptide fragments that enhance neurite outgrowth inhibition or inhibit abnormal neuron sprouting, for example, CNS neurons. For example, the present invention provides Nogo polypeptides and polypeptide fragments which inhibit abnormal neuron sprouting under conditions in which axonal growth is hyper or hypoactive. Thus, the Nogo polypeptides and polypeptide fragments of the invention are useful in treating injuries, diseases or disorders that can be alleviated by inhibiting abnormal neuronal sprouting or inhibiting neurite outgrowth.

Exemplary diseases, disorders or injuries include, but are not limited to, schizophrenia, bipolar disorder, obsessive-compulsive disorder (OCD), Attention Deficit Hyperactivity Disorder (ADHD), Downs Syndrome, and Alzheimer's disease.

Nogo and Nogo Receptor Polypeptides and Polypeptide Fragments

The present invention is directed to certain Nogo polypeptides and polypeptide fragments useful, e.g., for inhibiting neurite outgrowth or inhibiting abnormal neuronal sprouting. Typically, the Nogo polypeptides and polypeptide fragments of the invention act to enhance NgR1-mediated inhibition of neuronal survival, neurite outgrowth or axonal regeneration of central nervous system (CNS) neurons. The present invention is further directed to certain Nogo polypeptides and polypeptide fragments useful as a drug delivery machinery for targeting neurons or cells that specifically express NgR. The present invention is also directed to certain NgR polypeptides and polypeptide fragments for use in screening methods for potential drug candiates.

The human Nogo-A polypeptide is shown below as SEQ ID NO:2.

Full-Length Human Nogo-A (SEQ ID NO:2):

MEDLDQSPLVSSSDSPPRPQPAFKYQFVREPEDEEEEEEEEEEDEDEDLE
ELEVLERKPAAGLSAAPVPTAPAAGAPLMDFGNDFVPPAPRGPLPAAPPV
APERQPSWDPSPVSSTVPAPSPLSAAAVSPSKLPEDDEPPARPPPPPPAS
VSPQAEPVWTPPAPAPAAPPSTPAAPKRRGSSGSVDETLFALPAASEPVI
RSSAENMDLKEQPGNTISAGQEDFPSVLLETAASLPSLSPLSAASFKEHE
YLGNLSTVLPTEGTLQENVSEASKEVSEKAKTLLIDRDLTEFSELEYSEM
GSSFSVSPKAESAVIVANPREEIIVKNKDEEEKLVSNNILHNQQELPTAL
TKLVKEDEVVSSEKAKDSFNEKRVAVEAPMREEYADFKPFERVWEVKDSK
EDSDMLAAGGKIESNLESKVDKKCFADSLEQTNHEKDSESSNDDTSFPST
PEGIKDRSGAYITCAPFNPAATESIATNIFPLLGDPTSENKTDEKKIEEK
KAQIVTEKNTSTKTSNPFLVAAQDSETDYVTTDNLTKVTEEVVANMPEGL
TPDLVQEACESELNEVTGTKIAYETKMDLVQTSEVMQESLYPAAQLCPSF
EESEATPSPVLPDIVMEAPLNSAVPSAGASVIQPSSSPLEASSVNYESIK
HEPENIPPPYEEAMSVSLKKVSGIKEEIKEPENINAALQETEAPYISIAC
DLIKETKLSAEPAPDFSDYSEMAKVEQPVPDHSELVEDSSPDSEPVDLFS
DDSIPDVPQKQDETVMLVKESLTETSFESMIEYENKEKLSALPPEGGKPY
LESFKLSLDNTKDTLLPDEVSTLSKKEKIPLQMEELSTAVYSNDDLFISK
EAQIRETETFSDSSPIEIIDEFPTLISSKTDSFSKLAREYTDLEVSHKSE
IANAPDGAGSLPCTELPHDLSLKNIQPKVEEKISFSDDFSKNGSATSKVL
LLPPDVSALATQAEIESIVKPKVLVKEAEKKLPSDTEKEDRSPSAIFSAE
LSKTSVVDLLYWRDIKKTGVVFGASLFLLLSLTVFSIVSVTAYIALALLS
VTISFRIYKGVIQAIQKSDEGHPFRAYLESEVAISEELVQKYSNSALGHV
NCTIKELRRLFLVDDLVDSLKFAVLMWVFTYVGALFNGLTLLILALISLF
SVPVIYERHQAQIDHYLGLANKNVKDAMAKIQAKIPGLKRKAE

The full length Human NgR-1 is shown below as SEQ ID NO:4.

Full-Length Human NgR-1 (SEQ ID NO:4):

MKRASAGGSRLLAWVLWLQAWQVAAPCPGACVCYNEPKVTTSCPQQGLQA
VPVGIPAASQRFIFLHGNRISHVPAASFRACRNLTILWLHSNVLARIDAA
AFTGLALLEQLDLSDNAQLRSVDPATFHGLGRLHTLHLDRCGLQELGPGL
FRGLAALQYLYLQDNALQALPDDTFRDLGNLTHLFLHGNRISSVPERAFR
GLHSLDRLLLHQNRVAHVHPHAFRDLGRLMTLYLFANNLSALPTEALAPL
RALQYLRLNDNPWVCDCRARPLWAWLQKFRGSSSEVPCSLPQRLAGRDLK
RLAANDLQGCAVATGPYHPIWTGRATDEEPLGLPKCCQPDAADKASVLEP
GRPASAGNALKGRVPPGDSPPGNGSGPRHINDSPFGTLPGSAEPPLTAVR
PEGSEPPGFPTSGPRRRPGCSRKNRTRSHCRLGQAGSGGGGTGDSEGSGA
LPSLTCSLTPLGLALVLWTVLGPC

The full length Rat NgR-1 is shown below as SEQ ID NO:6.

Full-Length Rat NgR-1 (SEQ ID NO:6):

MKRASSGGSRLPTWVLWLQAWRVATPCPGACVCYKEPKVTTSRPQQGLQA
VPAGIPASSQRIFLHGNRISYVPAASFQSCRNLTILWLHSNALAGIDAAA
FTGLTLLEQLDLSDNAQLRVVDPTTFRGLGHLHTLHLDRCGLQELGPGLG
LAALQYLYLQDNNLQALPDNTFRDLGNLTHLFLHGNRIPSVPEHAFRGLH
SLDRLLLHQNHVARVHPHAFRDLGRLMTLYLFANNLSMLPAEVLVPLRSL
QYLRLNDNPWVCDCRARPLWAWLQKFRGSSSGVPSNLPQRLAGRDLKRLA
TSDLEGCAVASGPFRPFQTNQLTDEELLGLPKCCQPDAADKASVLEPGRP
ASVGNALKGRVPPGDTPPGNGSGPRHINDSPFGTLPGSAEPPLTALRPGG
SEPPGLPTTGPRRRPGCSRKNRTRSHCRLGQAGSGSSGTGDAEGSGALPA
LACSLAPL GLALVLWTVLGPC

In certain embodiments, the present invention provides an isolated polypeptide fragment of 30, 40, 50, 60, 70, 80, 90, or 100 residues or less, where the polypeptide fragment comprises an amino acid sequence at least 90% identical to a Nogo reference amino acid sequence where the polypeptide fragment binds NgR1. In particular embodiments, the polypeptide fragment is 30 residues or less. According to this embodiment, Nogo reference amino acid sequences include, but are not limited to amino acids 995 to 1013 of SEQ ID NO:2; amino acids 995 to 1014 of SEQ ID NO:2; amino acids 995 to 1015 of SEQ ID NO:2; amino acids 995 to 1016 of SEQ ID NO:2; amino acids 995 to 1017 of SEQ ID NO:2; amino acids 995 to 1018 of SEQ ID NO:2; amino acids 995 to 1019 of SEQ ID NO:2; amino acids 995 to 1020 of SEQ ID NO:2; amino acids 992 to 1018; amino acids 993 to 1018 of SEQ ID NO:2; and amino acids 994 to 1018 of SEQ ID NO:2. Polynucleotides encoding the polypeptide fragments, as well as vectors, and host cells comprising said polynucleotides are encompassed by the present invention. Polynucleotides, vectors, and host cells which express the polypeptide through operable association with expression control elements such as promoters are also included.

By “a Nogo reference amino acid sequence,” or “reference amino acid sequence” is meant the specified sequence without the introduction of any amino acid substitutions. As one of ordinary skill in the art would understand, if there are no substitutions, the “isolated polypeptide” of the invention comprises an amino acid sequence which is identical to the reference amino acid sequence.

Exemplary reference amino acid sequences according to this embodiment include amino acids 995 to 1013 of SEQ ID NO:2, and amino acids 995 to 1018 of SEQ ID NO:2.

Corresponding fragments of Nogo polypeptides or polypeptide fragments at least 70%, 75%, 80%, 85%, 90%, or 95% identical to SEQ ID NO:2 or fragments thereof described herein are also contemplated. As known in the art, “sequence identity” between two polypeptides is determined by comparing the amino acid sequence of one polypeptide to the sequence of a second polypeptide. When discussed herein, whether any particular polypeptide is at least about 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In one aspect, the invention includes a polypeptide comprising two or more polypeptide fragments as described above in a fusion protein, as well as fusion proteins comprising a polypeptide fragment as described above fused to a heterologous amino acid sequence. The invention further encompasses variants, analogs, or derivatives of polypeptide fragments as described above.

In one embodiment, the present invention provides an isolated polypeptide fragment of 200 residues or less, or 190, 180, 170, 160, 150, 140, 130 or 125 residues or less, comprising a first amino acid sequence that is at least 80%, 90%, or 95% identical to amino acids 995 to 1018 of SEQ ID NO:2, where the first amino acid sequence is linked, either directly or indirectly, to amino acids 1055 to 1086 of SEQ ID NO:2. In another embodiment, the polypeptide fragment comprises amino acids 995 to 1018 of SEQ ID NO:2 fused to amino acids 1055 to 1086 of SEQ ID NO:2. In other embodiments, the polypeptide fragment comprises an amino acid sequence at least 80%, 90%, or 95% identical to amino acids 950 to 1018, 960 to 1018, 970 to 1018, 980 to 1018, 990 to 1018, 995 to 1028, 995 to 1038, 995 to 1048, and 995 to 1054 of SEQ ID NO:2 where the polypeptide fragment is linked or fused to amino acids 1055 to 1086 of SEQ ID NO:2. In another embodiment, the polypeptide fragments bind NgR1. In certain embodiments, the polypeptide fragment enhances NgR-mediated neurite outgrowth inhibition. Rat NgR1 is also cotemplated in this embodiment. In another embodiment, the polypeptide fragment comprises SEQ ID NO:5. In a further embodiment, the polypeptide fragment consists essentially of SEQ ID NO:5. Polynucleotides encoding the polypeptide fragments, as well as vectors, and host cells comprising said polynucleotides are encompassed by the present invention. Polynucleotides, vectors, and host cells which express the polypeptide through operable association with expression control elements such as promoters are also included.

The 24-32 fusion peptide is shown below as SEQ ID NO:5

Amino-Nogo24 fused to NEP32 (SEQ ID NO:5):

IFSAELSKTSVVDLLYWRDIKKTGGRIYKGVIQAIQKSDEGHPFRAYLES
EVAISEE

In another embodiment, the present invention provides NgR1 polypeptide variants with altered ligand binding characterisitics. For example, the present invention provides an isolated polypeptide comprising amino acids 27 to 473 of SEQ ID NO:4, i.e., the mature NgR1 polypeptide, except for amino acid substitutions at the amino acid positions selected from the group consisting of: (a) amino acids 67, 68, and 71 of SEQ ID NO:4; (b) amino acids 111, 113, and 114 of SEQ ID NO:4; (c) amino acids 133 and 136 of SEQ ID NO:4; (d) amino acids 158, 160, 182 and 186 of SEQ ID NO:4; (e) amino acid 163 of SEQ ID NO:4; and (f) amino acids 232 and 234 of SEQ ID NO:4. In certain embodiments, the polypeptide of the present invention does not bind any of Nogo-66, OMgp, Mag, or Lingo-1.

In another embodiment, the present invention provides an isolated polypeptide comprising amino acids 27 to 473 of SEQ ID NO:4 and amino acid substitutions at least the amino acid positions selected from the group consisting of: (a) amino acids 78 and 81 of SEQ ID NO:4; (b) amino acids 87 and 89 of SEQ ID NO:4; (c) amino acids 89 and 90 of SEQ ID NO:4; (d) amino acids 95 and 97 of SEQ ID NO:4; (e) amino acid 108 of SEQ ID NO:4; (f) amino acids 117, 119 and 120 of SEQ ID NO:4; (g) amino acid 13 of SEQ ID NO:4; (h) amino acid 210 of SEQ ID NO:4; and (i) amino acids 256 and 259 of SEQ ID NO:4. In certain embodiments, the polypeptide of the present invention binds to at least one but not all of Nogo-66, OMgp, Mag, or Lingo-1. Similar NgR1 polypeptide variants of rat or mouse NgR1 are also contemplated. Polynucleotides encoding the polypeptide fragments, as well as vectors, and host cells comprising said polynucleotides are encompassed by the present invention. Polynucleotides, vectors, and host cells which express the polypeptide through operable association with expression control elements such as promoters are also included.

Additional embodiments that are envisioned include polynucleotides that encode the polypeptides or fragments thereof of the present invention, and host cells or vestors that express the polypeptides or fragments thereof of the present invention.

The amino acid residues in the polypeptides or fragments thereof of the present invention may be substituted with any heterologous amino acid. In certain embodiments, the amino acid is substituted with a small uncharged amino acid which is least likely to alter the three dimensional conformation of the polypeptide, e.g., alanine, serine, threonine, preferably alanine. In other embodiments, the amino acids are substituted with alanine.

In the present invention, a polypeptide or fragments thereof can be composed of amino acids joined to each other by peptide bonds or modified peptide bonds, i.e., peptide isosteres, and may contain amino acids other than the 20 gene-encoded amino acids (e.g. non-naturally occurring amino acids). The polypeptides of the present invention may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W.H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).).

Polypeptides or fragments thereof described herein may be cyclic. Cyclization of the polypeptides reduces the conformational freedom of linear peptides and results in a more structurally constrained molecule. Many methods of peptide cyclization are known in the art. For example, “backbone to backbone” cyclization by the formation of an amide bond between the N-terminal and the C-terminal amino acid residues of the peptide. The “backbone to backbone” cyclization method includes the formation of disulfide bridges between two ω-thio amino acid residues (e.g. cysteine, homocysteine). Certain peptides of the present invention include modifications on the N- and C-terminus of the peptide to form a cyclic polypeptide. Such modifications include, but are not limited, to cysteine residues, acetylated cysteine residues, cysteine residues with a NH2 moiety and biotin. Other methods of peptide cyclization are described in Li & Roller, Curr. Top. Med. Chem. 3:325-341 (2002), which is incorporated by reference herein in its entirety.

In certain methods of the present invention, polypeptides or fragments thereof of the present invention can be administered directly as a preformed polypeptide, or indirectly through a nucleic acid vector. In some embodiments of the invention, a polypeptide or fragment thereof of the present invention is administered in a treatment method that includes: (1) transforming or transfecting an implantable host cell with a nucleic acid, e.g., a vector, that expresses a polypeptide or fragment thereof of the present invention; and (2) implanting the transformed host cell into a mammal, at the site of a disease, disorder or injury. In some embodiments of the invention, the implantable host cell is removed from a mammal, temporarily cultured, transformed or transfected with an isolated nucleic acid encoding a polypeptide or fragment thereof of the present invention, and implanted back into the same mammal from which it was removed. The cell can be, but is not required to be, removed from the same site at which it is implanted. Such embodiments, sometimes known as ex vivo gene therapy, can provide a continuous supply of the polypeptide or fragment thereof of the present invention, localized at the site of action, for a limited period of time.

Additional exemplary polypeptides or fragments thereof of the present invention and methods and materials for obtaining these molecules for practicing the present invention are described below.

Fusion Proteins and Conjugated Polypeptides

Some embodiments of the invention involve the use of a polypeptide of the present invention that is not the full-length protein, e.g., polypeptide fragments, fused to a heterologous polypeptide moiety to form a fusion protein. Such fusion proteins can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous moiety can be inert or biologically active. Also, it can be chosen to be stably fused to the polypeptide moiety of the invention or to be cleavable, in vitro or in vivo. Heterologous moieties to accomplish these other objectives are known in the art.

As an alternative to expression of a fusion protein, a chosen heterologous moiety can be preformed and chemically conjugated to the polypeptide moiety of the present invention. In most cases, a chosen heterologous moiety will function similarly, whether fused or conjugated to the polypeptide moiety. Therefore, in the following discussion of heterologous amino acid sequences, unless otherwise noted, it is to be understood that the heterologous sequence can be joined to the polypeptide moiety in the form of a fusion protein or as a chemical conjugate.

Pharmacologically active polypeptides such as the polypeptides or fragments thereof of the present invention may exhibit rapid in vivo clearance, necessitating large doses to achieve therapeutically effective concentrations in the body. In addition, polypeptides smaller than about 60 kDa potentially undergo glomerular filtration, which sometimes leads to nephrotoxicity. Fusion or conjugation of relatively small polypeptides can be employed to reduce or avoid the risk of such nephrotoxicity. Various heterologous amino acid sequences, i.e., polypeptide moieties or “carriers,” for increasing the in vivo stability, i.e., serum half-life, of therapeutic polypeptides are known. Examples include serum albumins such as, e.g., bovine serum albumin (BSA) or human serum albumin (HSA).

Due to its long half-life, wide in vivo distribution, and lack of enzymatic or immunological function, essentially full-length human serum albumin (HSA), or an HSA fragment, is commonly used as a heterologous moiety. Through application of methods and materials such as those taught in Yeh et al., Proc. Natl. Acad. Sci. USA, 89:1904-08 (1992) and Syed et al., Blood 89:3243-52 (1997), HSA can be used to form a fusion protein or polypeptide conjugate that displays pharmacological activity by virtue of the polypeptide moiety while displaying significantly increased in vivo stability, e.g., 10-fold to 100-fold higher. The C-terminus of the HSA can be fused to the N-terminus of the polypeptide moiety. Since HSA is a naturally secreted protein, the HSA signal sequence can be exploited to obtain secretion of the fusion protein into the cell culture medium when the fusion protein is produced in a eukaryotic, e.g., mammalian, expression system.

Some embodiments of the invention employ a polypeptide moiety fused to a hinge and Fc region, i.e., the C-terminal portion of an Ig heavy chain constant region. Potential advantages of a polypeptide-Fc fusion include solubility, in vivo stability, and multivalency, e.g., dimerization. The Fc region used can be an IgA, IgD, or IgG Fc region (hinge-CH2-CH3). Alternatively, it can be an IgE or IgM Fc region (hinge-CH2-CH3-CH4). An IgG Fc region is generally used, e.g., an IgG1 Fc region or IgG4 Fc region. Materials and methods for constructing and expressing DNA encoding Fc fusions are known in the art and can be applied to obtain fusions without undue experimentation. Some embodiments of the invention employ a fusion protein such as those described in Capon et al., U.S. Pat. Nos. 5,428,130 and 5,565,335.

The signal sequence is a polynucleotide that encodes an amino acid sequence that initiates transport of a protein across the membrane of the endoplasmic reticulum. Signal sequences useful for constructing an immunofusin include antibody light chain signal sequences, e.g., antibody 14.18 (Gillies et al., J. Immunol. Meth., 125:191-202 (1989)), antibody heavy chain signal sequences, e.g., the MOPC141 antibody heavy chain signal sequence (Sakano et al., Nature 286:5774 (1980)). Alternatively, other signal sequences can be used. See, e.g., Watson, Nucl. Acids Res. 12:5145 (1984). The signal peptide is usually cleaved in the lumen of the endoplasmic reticulum by signal peptidases. This results in the secretion of a immunofasin protein containing the Fc region and the polypeptide moiety.

In some embodiments, the DNA sequence may encode a proteolytic cleavage site between the secretion cassette and the polypeptide moiety. Such a cleavage site may provide, e.g., for the proteolytic cleavage of the encoded fusion protein, thus separating the Fc domain from the target protein. Useful proteolytic cleavage sites include amino acid sequences recognized by proteolytic enzymes such as trypsin, plasmin, thrombin, factor Xa, or enterokinase K.

The secretion cassette can be incorporated into a replicable expression vector. Useful vectors include linear nucleic acids, plasmids, phagemids, cosmids and the like. An exemplary expression vector is pdC, in which the transcription of the immunofusin DNA is placed under the control of the enhancer and promoter of the human cytomegalovirus. See, e.g., Lo et al., Biochim. Biophys. Acta 1088:712 (1991); and Lo et al., Protein Engineering 11:495-500 (1998). An appropriate host cell can be transformed or transfected with a DNA that encodes a polypeptide or fragment thereof of the present invention and used for the expression and secretion of the polypeptide. Host cells that are typically used include immortal hybridoma cells, myeloma cells, 293 cells, Chinese hamster ovary (CHO) cells, Hela cells, and COS cells.

Fully intact, wild-type Fc regions display effector functions that normally are unnecessary and undesired in an Fc fusion protein used in the methods of the present invention. Therefore, certain binding sites typically are deleted from the Fc region during the construction of the secretion cassette. For example, since coexpression with the light chain is unnecessary, the binding site for the heavy chain binding protein, Bip (Hendershot et al., Immunol. Today 8:111-14 (1987)), is deleted from the CH2 domain of the Fc region of IgE, such that this site does not interfere with the efficient secretion of the immunofusin. Transmembrane domain sequences, such as those present in IgM, also are generally deleted.

The IgG1 Fe region is most often used. Alternatively, the Fc region of the other subclasses of immunoglobulin gamma (gamma-2, gamma-3 and gamma-4) can be used in the secretion cassette. The IgG1 Fc region of immunoglobulin gamma-1 is generally used in the secretion cassette and includes at least part of the hinge region, the CH2 region, and the CH3 region. In some embodiments, the Fc region of immunoglobulin gamma-1 is a CH2-deleted-Fc, which includes part of the hinge region and the CH3 region, but not the CH2 region. A CH2-deleted-Fc has been described by Gillies et al., Hum. Antibod. Hybridomas 1:47 (1990). In some embodiments, the Fc region of one of IgA, IgD, IgE, or IgM, is used.

Polypeptide-moiety-Fc fusion proteins can be constructed in several different configurations. In one configuration the C-terminus of the polypeptide moiety is fused directly to the N-terminus of the Fc hinge moiety. In a slightly different configuration, a short polypeptide, e.g., 2-10 amino acids, is incorporated into the fusion between the N-terminus of the polypeptide moiety and the C-terminus of the Fc moiety. Such a linker provides conformational flexibility, which may improve biological activity in some circumstances. If a sufficient portion of the hinge region is retained in the Fc moiety, the polypeptide-moiety-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

Any of a number of cross-linkers that contain a corresponding amino-reactive group and thiol-reactive group can be used to link a polypeptide or fragment thereof of the present invention to serum albumin. Examples of suitable linkers include amine reactive cross-linkers that insert a thiol-reactive maleimide, e.g., SMCC, AMAS, BMPS, MBS, EMCS, SMPB, SMPH, KMUS, and GMBS. Other suitable linkers insert a thiol-reactive haloacetate group, e.g., SBAP, SIA, SIAB. Linkers that provide a protected or non-protected thiol for reaction with sulfhydryl groups to product a reducible linkage include SPDP, SMPT, SATA, and SATP. Such reagents are commercially available (e.g., Pierce Chemical Company, Rockford, Ill.).

Conjugation does not have to involve the N-terminus of a polypeptide or fragment thereof of the present invention or the thiol moiety on serum albumin. For example, polypeptide-albumin fusions can be obtained using genetic engineering techniques, wherein the polypeptide moiety is fused to the serum albumin gene at its N-terminus, C-terminus, or both.

Polypeptides or fragments thereof of the present invention can be fused to a polypeptide tag. The term “polypeptide tag,” as used herein, is intended to mean any sequence of amino acids that can be attached to, connected to, or linked to a polypeptide or fragment thereof of the present invention and that can be used to identify, purify, concentrate or isolate the polypeptide or fragment thereof. The attachment of the polypeptide tag to the polypeptide or fragment thereof may occur, e.g., by constructing a nucleic acid molecule that comprises: (a) a nucleic acid sequence that encodes the polypeptide tag, and (b) a nucleic acid sequence that encodes a polypeptide or fragment thereof of the present invention. Exemplary polypeptide tags include, e.g., amino acid sequences that are capable of being post-translationally modified, e.g., amino acid sequences that are biotinylated. Other exemplary polypeptide tags include, e.g., amino acid sequences that are capable of being recognized and/or bound by an antibody (or fragment thereof) or other specific binding reagent. Polypeptide tags that are capable of being recognized by an antibody (or fragment thereof) or other specific binding reagent include, e.g., those that are known in the art as “epitope tags.” An epitope tag may be a natural or an artificial epitope tag. Natural and artificial epitope tags are known in the art, including, e.g., artificial epitopes such as FLAG, Strep, or poly-histidine peptides. FLAG peptides include the sequence Asp-Tyr-Lys-Asp-Asp-Asp-Asp-Lys (SEQ ID NO:______) or Asp-Tyr-Lys-Asp-Glu-Asp-Asp-Lys (SEQ ID NO:______) (Einhauer, A. and Jungbauer, A., J. Biochem. Biophlys. Methods 49:1-3:455-465 (2001)). The Strep epitope has the sequence Ala-Trp-Arg-His-Pro-Gln-Phe-Gly-Gly (SEQ ID NO:______). The VSV-G epitope can also be used and has the sequence Tyr-Thr-Asp-Ile-Glu-Met-Asn-Arg-Leu-Gly-Lys (SEQ ID NO:______). Another artificial epitope is a poly-His sequence having six histidine residues (His-His-His-His-His-His (SEQ ID NO:______). Naturally-occurring epitopes include the influenza virus hemagglutinin (HA) sequence Tyr-Pro-Tyr-Asp-Val-Pro-Asp-Tyr-Ala-Ile-Glu-Gly-Arg (SEQ ID NO:______) recognized by the monoclonal antibody 12CA5 (Murray et al., Anal. Biochem. 229:170-179 (1995)) and the eleven amino acid sequence from human c-myc (Myc) recognized by the monoclonal antibody 9E10 (Glu-Gln-Lys-Leu-Leu-Ser-Glu-Glu-Asp-Leu-Asn (SEQ ID NO:______) (Manstein et al., Gene 162:129-134 (1995)). Another useful epitope is the tripeptide Glu-Glu-Phe which is recognized by the monoclonal antibody YL 1/2. (Stammers et al. FEBS Lett. 283:298-302 (1991)).

In certain embodiments, the polypeptide or fragment thereof of the present invention and the polypeptide tag may be connected via a linking amino acid sequence. As used herein, a “linking amino acid sequence” may be an amino acid sequence that is capable of being recognized and/or cleaved by one or more proteases. Amino acid sequences that can be recognized and/or cleaved by one or more proteases are known in the art. Exemplary amino acid sequences are those that are recognized by the following proteases: factor VIIa, factor IXa, factor Xa, APC, t-PA, u-PA, trypsin, chymotrypsin, enterokinase, pepsin, cathepsin B,H,L,S,D, cathepsin G, renin, angiotensin converting enzyme, matrix metalloproteases (collagenases, stromelysins, gelatinases), macrophage elastase, Cir, and Cis. The amino acid sequences that are recognized by the aforementioned proteases are known in the art. Exemplary sequences recognized by certain proteases can be found, e.g., in U.S. Pat. No. 5,811,252.

Polypeptide tags can facilitate purification using commercially available chromatography media.

In some embodiments of the invention, a polypeptide fusion construct is used to enhance the production of a polypeptide moiety of the present invention in bacteria. In such constructs a bacterial protein normally expressed and/or secreted at a high level is employed as the N-terminal fusion partner of a polypeptide or fragment thereof of the present invention. See, e.g., Smith et al., Gene 67:31 (1988); Hopp et al., Biotechnology 6:1204 (1988); La Vallie et al., Biotechnology 11:187 (1993).

By fusing a polypeptide moiety of the present invention at the amino and carboxy termini of a suitable fusion partner, bivalent or tetravalent forms of a polypeptide or fragment thereof of the present invention can be obtained. For example, a polypeptide moiety of the present invention can be fused to the amino and carboxy termini of an Ig moiety to produce a bivalent monomeric polypeptide containing two polypeptide moieties of the present invention. Upon dimerization of two of these monomers, by virtue of the Ig moiety, a tetravalent form of a polypeptide of the present invention is obtained. Such multivalent forms can be used to achieve increased binding affinity for the target. Multivalent forms of a polypeptide or fragment thereof of the present invention also can be obtained by placing polypeptide moieties of the present invention in tandem to form concatamers, which can be employed alone or fused to a fusion partner such as Ig or HSA.

Conjugated Polymers (Other than Polypeptides)

Some embodiments of the invention involve a polypeptide or fragment thereof of the present invention wherein one or more polymers are conjugated (covalently linked) to the polypeptide or fragment thereof of the present invention. Examples of polymers suitable for such conjugation include polypeptides (discussed above), sugar polymers and polyalkylene glycol chains. Typically, but not necessarily, a polymer is conjugated to the polypeptide or fragment thereof of the present invention for the purpose of improving one or more of the following: solubility, stability, or bioavailability.

The class of polymer generally used for conjugation to a polypeptide or fragment thereof of the present invention is a polyalkylene glycol. Polyethylene glycol (PEG) is most frequently used. PEG moieties, e.g., 1, 2, 3, 4 or 5 PEG polymers, can be conjugated to each Polypeptide or fragment thereof of the present invention to increase serum half life, as compared to the polypeptide or fragment thereof of the present invention alone. PEG moieties are non-antigenic and essentially biologically inert. PEG moieties used in the practice of the invention may be branched or unbranched.

The number of PEG moieties attached to the polypeptide or fragment thereof of the present invention and the molecular weight of the individual PEG chains can vary. In general, the higher the molecular weight of the polymer, the fewer polymer chains attached to the polypeptide. Usually, the total polymer mass attached to a polypeptide or fragment thereof of the present invention is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to the polypeptide or fragment thereof of the present invention through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the polypeptide or fragment thereof of the present invention. Free carboxylic groups, suitably activated carbonyl groups, hydroxyl, guanidyl, imidazole, oxidized carbohydrate moieties and mercapto groups of the polypeptide or fragment thereof of the present invention (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the polypeptide moiety of the present invention. Preferably, at least 50% of the biological activity (as demonstrated, e.g., in any of the assays described herein or known in the art) of the polypeptide or fragment thereof of the present invention is retained, and most preferably nearly 100% is retained.

The polymer can be conjugated to the polypeptide or fragment thereof of the present invention using conventional chemistry. For example, a polyalkylene glycol moiety can be coupled to a lysine epsilon amino group of the polypeptide or fragment thereof of the present invention. Linkage to the lysine side chain can be performed with an N-hydroxylsuccinimide (NHS) active ester such as PEG succinimidyl succinate (SS-PEG) and succinimidyl propionate (SPA-PEG). Suitable polyalkylene glycol moieties include, e.g., carboxymethyl-NHS and norleucine-NHS, SC. These reagents are commercially available. Additional amine-reactive PEG linkers can be substituted for the succinimidyl moiety. These include, e.g., isothiocyanates, nitrophenylcarbonates (PNP), epoxides, benzotriazole carbonates, SC-PEG, tresylate, aldehyde, epoxide, carbonylimidazole and PNP carbonate. Conditions are usually optimized to maximize the selectivity and extent of reaction. Such optimization of reaction conditions is within ordinary skill in the art.

PEGylation can be carried out by any of the PEGylation reactions known in the art. See, e.g., Focus on Growth Factors, 3: 4-10, 1992 and European patent applications EP 0 154 316 and EP 0 401 384. PEGylation may be carried out using an acylation reaction or an alkylation reaction with a reactive polyethylene glycol molecule (or an analogous reactive water-soluble polymer).

PEGylation by acylation generally involves reacting an active ester derivative of polyethylene glycol. Any reactive PEG molecule can be employed in the PEGylation. PEG esterified to N-hydroxysuccinimide (NHS) is a frequently used activated PEG ester. As used herein, “acylation” includes without limitation the following types of linkages between the therapeutic protein and a water-soluble polymer such as PEG: amide, carbamate, urethane, and the like. See, e.g., Bioconjugate Chem. 5: 133-140, 1994. Reaction parameters are generally selected to avoid temperature, solvent, and pH conditions that would damage or inactivate the polypeptide or fragment thereof of the present invention.

Generally, the connecting linkage is an amide and typically at least 95% of the resulting product is mono-, di- or tri-PEGylated. However, some species with higher degrees of PEGylation may be formed in amounts depending on the specific reaction conditions used. Optionally, purified PEGylated species are separated from the mixture, particularly unreacted species, by conventional purification methods, including, e.g., dialysis, salting-out, ultrafiltration, ion-exchange chromatography, gel filtration chromatography, hydrophobic exchange chromatography, and electrophoresis.

PEGylation by alkylation generally involves reacting a terminal aldehyde derivative of PEG with a polypeptide or fragment thereof of the present invention in the presence of a reducing agent. In addition, one can manipulate the reaction conditions to favor PEGylation substantially only at the N-terminal amino group of the polypeptide or fragment thereof of the present invention, i.e. a mono-PEGylated protein. In either case of mono-PEGylation or poly-PEGylation, the PEG groups are typically attached to the protein via a —CH2-NH— group. With particular reference to the —CH2— group, this type of linkage is known as an “alkyl” linkage.

Derivatization via reductive alkylation to produce an N-terminally targeted mono-PEGylated product exploits differential reactivity of different types of primary amino groups (lysine versus the N-terminal) available for derivatization. The reaction is performed at a pH that allows one to take advantage of the pKa differences between the epsilon-amino groups of the lysine residues and that of the N-terminal amino group of the protein. By such selective derivatization, attachment of a water-soluble polymer that contains a reactive group, such as an aldehyde, to a protein is controlled: the conjugation with the polymer takes place predominantly at the N-terminus of the protein and no significant modification of other reactive groups, such as the lysine side chain amino groups, occurs.

The polymer molecules used in both the acylation and alkylation approaches are selected from among water-soluble polymers. The polymer selected is typically modified to have a single reactive group, such as an active ester for acylation or an aldehyde for alkylation, so that the degree of polymerization may be controlled as provided for in the present methods. An exemplary reactive PEG aldehyde is polyethylene glycol propionaldehyde, which is water stable, or mono C1-C10 alkoxy or aryloxy derivatives thereof (see, e.g., Harris et al., U.S. Pat. No. 5,252,714). The polymer may be branched or unbranched. For the acylation reactions, the polymer(s) selected typically have a single reactive ester group. For reductive alkylation, the polymer(s) selected typically have a single reactive aldehyde group. Generally, the water-soluble polymer will not be selected from naturally occurring glycosyl residues, because these are usually made more conveniently by mammalian recombinant expression systems.

Methods for preparing PEGylated polypeptides or fragments thereof of the present invention generally includes the steps of (a) reacting a polypeptide or fragment thereof of the present invention with polyethylene glycol (such as a reactive ester or aldehyde derivative of PEG) under conditions whereby the molecule becomes attached to one or more PEG groups, and (b) obtaining the reaction product(s). In general, the optimal reaction conditions for the acylation reactions will be determined case-by-case based on known parameters and the desired result. For example, a larger the ratio of PEG to protein, generally leads to a greater the percentage of poly-PEGylated product.

Reductive alkylation to produce a substantially homogeneous population of mono-polymer/polypeptide or fragment thereof of the present invention generally includes the steps of: (a) reacting a polypeptide or fragment thereof of the present invention with a reactive PEG molecule under reductive alkylation conditions, at a pH suitable to permit selective modification of the N-terminal amino group of the polypeptide or fragment thereof of the present invention; and (b) obtaining the reaction product(s).

For a substantially homogeneous population of mono-polymer/polypeptide or fragment thereof of the present invention, the reductive alkylation reaction conditions are those that permit the selective attachment of the water-soluble polymer moiety to the N-terminus of a polypeptide or fragment thereof of the present invention. Such reaction conditions generally provide for pKa differences between the lysine side chain amino groups and the N-terminal amino group. For purposes of the present invention, the pH is generally in the range of 3-9, typically 3-6.

Polypeptides or fragments thereof of the present invention can include a tag, e.g., a moiety that can be subsequently released by proteolysis. Thus, the lysine moiety can be selectively modified by first reacting a His-tag modified with a low-molecular-weight linker such as Traut's reagent (Pierce Chemical Company, Rockford, Ill.) which will react with both the lysine and N-terminus, and then releasing the His tag. The polypeptide will then contain a free SH group that can be selectively modified with a PEG containing a thiol-reactive head group such as a maleimide group, a vinylsulfone group, a haloacetate group, or a free or protected SH.

Traut's reagent can be replaced with any linker that will set up a specific site for PEG attachment. For example, Traut's reagent can be replaced with SPDP, SMPT, SATA, or SATP (Pierce Chemical Company, Rockford, Ill.). Similarly one could react the protein with an amine-reactive linker that inserts a maleimide (for example SMCC, AMAS, BMPS, MBS, EMCS, SMPB, SMPH, KMUS, or GMBS), a haloacetate group (SBAP, SIA, SIAB), or a vinylsulfone group and react the resulting product with a PEG that contains a free SH.

In some embodiments, the polyalkylene glycol moiety is coupled to a cysteine group of the polypeptide or fragment thereof of the present invention. Coupling can be effected using, e.g., a maleimide group, a vinylsulfone group, a haloacetate group, or a thiol group.

Optionally, the polypeptide or fragment thereof of the present invention is conjugated to the polyethylene-glycol moiety through a labile bond. The labile bond can be cleaved in, e.g., biochemical hydrolysis, proteolysis, or sulfhydryl cleavage. For example, the bond can be cleaved under in vivo (physiological) conditions.

The reactions may take place by any suitable method used for reacting biologically active materials with inert polymers, generally at about pH 5-8, e.g., pH 5, 6, 7, or 8, if the reactive groups are on the alpha amino group at the N-terminus. Generally the process involves preparing an activated polymer and thereafter reacting the protein with the activated polymer to produce the soluble protein suitable for formulation.

The polypeptides or fragments thereof of the present invention, in certain embodiments, are soluble polypeptides. Methods for making a polypeptide soluble or improving the solubility of a polypeptide are well known in the art.

Polynucleotides

The present invention also includes isolated polynucleotides that encode any one of the polypeptides or fragments thereof of the present invention. The invention also includes polynucleotides that hybridize under moderately stringent or high stringency conditions to the noncoding strand, or complement, of a polynucleotide that encodes any one of the polypeptides of the invention. Stringent conditions are known to those skilled in the art and can be found in CURRENT PROTOCOLS IN MOLECULAR BIOLOGY, John Wiley & Sons, N.Y. (1989), 6.3.1-6.3.6.

The human Nogo-A polynucleotide is shown below as SEQ ID NO: 1.

Full-Length Human Nogo-A (SEQ ID NO:1) encoded by nucleotide 135 to nucleotide 3710:

caccacagta ggtccctcgg ctcagtcggc ccagcccctc tcagtcctcc ccaaccccca caaccgcccg cggctctgag
acgcggcccc ggcggcggcg gcagcagctg cagcatcatc tccaccctcc agccatggaa gacctggacc agtctcctct
ggtctcgtcc tcggacagcc caccccggcc gcagcccgcg ttcaagtacc agttcgtgag ggagcccgag gacgaggagg
aagaagagga ggaggaagag gaggacgagg acgaagacct ggaggagctg gaggtgctgg agaggaagcc cgccgccggg
ctgtccgcgg ccccagtgcc caccgcccct gccgccggcg cgcccctgat ggacttcgga aatgacttcg tgccgccggc
gccccgggga cccctgccgg ccgctccccc cgtcgccccg gagcggcagc cgtcttggga cccgagcccg gtgtcgtcga
ccgtgcccgc gccatccccg ctgtctgctg ccgcagtctc gccctccaag ctccctgagg acgacgagcc tccggcccgg
cctccccctc ctcccccggc cagcgtgagc ccccaggcag agcccgtgtg gaccccgcca gccccggctc ccgccgcgcc
cccctccacc ccggccgcgc ccaagcgcag gggctcctcg ggctcagtgg atgagaccct ttttgctctt cctgctgcat
ctgagcctgt gatacgctcc tctgcagaaa atatggactt gaaggagcag ccaggtaaca ctatttcggc tggtcaagag
gatttcccat ctgtcctgct tgaaactgct gcttctcttc cttctctgtc tcctctctca gccgcttctt tcaaagaaca
tgaatacctt ggtaatttgt caacagtatt acccactgaa ggaacacttc aagaaaatgt cagtgaagct tctaaagagg
tctcagagaa ggcaaaaact ctactcatag atagagattt aacagagttt tcagaattag aatactcaga aatgggatca
tcgttcagtg tctctccaaa agcagaatct gccgtaatag tagcaaatcc tagggaagaa ataatcgtga aaaataaaga
tgaagaagag aagttagtta gtaataacat ccttcataat caacaagagt tacctacagc tcttactaaa ttggttaaag
aggatgaagt tgtgtcttca gaaaaagcaa aagacagttt taatgaaaag agagttgcag tggaagctcc tatgagggag
gaatatgcag acttcaaacc atttgagcga gtatgggaag tgaaagatag taaggaagat agtgatatgt tggctgctgg
aggtaaaatc gagagcaact tggaaagtaa agtggataaa aaatgttttg cagatagcct tgagcaaact aatcacgaaa
aagatagtga gagtagtaat gatgatactt ctttccccag tacgccagaa ggtataaagg atcgttcagg agcatatatc
acatgtgctc cctttaaccc agcagcaact gagagcattg caacaaacat ttttcctttg ttaggagatc ctacttcaga
aaataagacc gatgaaaaaa aaatagaaga aaagaaggcc caaatagtaa cagagaagaa tactagcacc aaaacatcaa
acccttttct tgtagcagca caggattctg agacagatta tgtcacaaca gataatttaa caaaggtgac tgaggaagtc
gtggcaaaca tgcctgaagg cctgactcca gatttagtac aggaagcatg tgaaagtgaa ttgaatgaag ttactggtac
aaagattgct tatgaaacaa aaatggactt ggttcaaaca tcagaagtta tgcaagagtc actctatcct gcagcacagc
tttgcccatc atttgaagag tcagaagcta ctccttcacc agttttgcct gacattgtta tggaagcacc attgaattct
gcagttccta gtgctggtgc ttccgtgata cagcccagct catcaccatt agaagcttct tcagttaatt atgaaagcat
aaaacatgag cctgaaaacc ccccaccata tgaagaggcc atgagtgtat cactaaaaaa agtatcagga ataaaggaag
aaattaaaga gcctgaaaat attaatgcag ctcttcaaga aacagaagct ccttatatat ctattgcatg tgatttaatt
aaagaaacaa agctttctgc tgaaccagct ccggatttct ctgattattc agaaatggca aaagttgaac agccagtgcc
tgatcattct gagctagttg aagattcctc acctgattct gaaccagttg acttatttag tgatgattca atacctgacg
ttccacaaaa acaagatgaa actgtgatgc ttgtgaaaga aagtctcact gagacttcat ttgagtcaat gatagaatat
gaaaataagg aaaaactcag tgctttgcca cctgagggag gaaagccata tttggaatct tttaagctca gtttagataa
cacaaaagat accctgttac ctgatgaagt ttcaacattg agcaaaaagg agaaaattcc tttgcagatg gaggagctca
gtactgcagt ttattcaaat gatgacttat ttatttctaa ggaagcacag ataagagaaa ctgaaacgtt ttcagattca
tctccaattg aaattataga tgagttccct acattgatca gttctaaaac tgattcattt tctaaattag ccagggaata
tactgaccta gaagtatccc acaaaagtga aattgctaat gccccggatg gagctgggtc attgccttgc acagaattgc
cccatgacct ttctttgaag aacatacaac ccaaagttga agagaaaatc agtttctcag atgacttttc taaaaatggg
tctgctacat caaaggtgct cttattgcct ccagatgttt ctgctttggc cactcaagca gagatagaga gcatagttaa
acccaaagtt cttgtgaaag aagctgagaa aaaacttcct tccgatacag aaaaagagga cagatcacca tctgctatat
tttcagcaga gctgagtaaa acttcagttg ttgacctcct gtactggaga gacattaaga agactggagt ggtgtttggt
gccagcctat tcctgctgct ttcattgaca gtattcagca ttgtgagcgt aacagcctac attgccttgg ccctgctctc
tgtgaccatc agctttagga tatacaaggg tgtgatccaa gctatccaga aatcagatga aggccaccca ttcagggcat
atctggaatc tgaagttgct atatctgagg agttggttca gaagtacagt aattctgctc ttggtcatgt gaactgcacg
ataaaggaac tcaggcgcct cttcttagtt gatgatttag ttgattctct gaagtttgca gtgttgatgt gggtatttac
ctatgttggt gccttgttta atggtctgac actactgatt ttggctctca tttcactctt cagtgttcct gttatttatg
aacggcatca ggcacagata gatcattatc taggacttgc aaataagaat gttaaagatg ctatggctaa aatccaagca
aaaatccctg gattgaagcg caaagctgaa tgaaaacgcc caaaataatt agtaggagtt catctttaaa ggggatattc
atttgattat acgggggagg gtcagggaag aacgaacctt gacgttgcag tgcagtttca cagatcgttg ttagatcttt
atttttagcc atgcactgtt gtgaggaaaa attacctgtc ttgactgcca tgtgttcatc atcttaagta ttgtaagctg
ctatgtatgg atttaaaccg taatcatatc tttttcctat ctgaggcact ggtggaataa aaaacctgta tattttactt
tgttgcagat agtcttgccg catcttggca agttgcagag atggtggagc tag

The human Nogo receptor-1 polynucleotide is shown below as SEQ ID NO:3.

Full-Length Human Nogo receptor-1 (SEQ ID NO:3) encoded by nucleotide 13 to nucleotide 1422:

ccaaccccta cgatgaagag ggcgtccgct ggagggagcc ggctgctggc atgggtgctg tggctgcagg
cctggcaggt ggcagcccca tgcccaggtg cctgcgtatg ctacaatgag cccaaggtga cgacaagctg
cccccagcag ggcctgcagg ctgtgcccgt gggcatccct gctgccagcc agcgcatctt cctgcacggc
aaccgcatct cgcatgtgcc agctgccagc ttccgtgcct gccgcaacct caccatcctg tggctgcact
cgaatgtgct ggcccgaatt gatgcggctg ccttcactgg cctggccctc ctggagcagc tggacctcag
cgataatgca cagctccggt ctgtggaccc tgccacattc cacggcctgg gccgcctaca cacgctgcac
ctggaccgct gcggcctgca ggagctgggc ccggggctgt tccgcggcct ggctgccctg cagtacctct
acctgcagga caacgcgctg caggcactgc ctgatgacac cttccgcgac ctgggcaacc tcacacacct
cttcctgcac ggcaaccgca tctccagcgt gcccgagcgc gccttccgtg ggctgcacag cctcgaccgt
ctcctactgc accagaaccg cgtggcccat gtgcacccgc atgccttccg tgaccttggc cgcctcatga
cactctatct gtttgccaac aatctatcag cgctgcccac tgaggccctg gcccccctgc gtgccctgca
gtacctgagg ctcaacgaca acccctgggt gtgtgactgc cgggcacgcc cactctgggc ctggctgcag
aagttccgcg gctcctcctc cgaggtgccc tgcagcctcc cgcaacgcct ggctggccgt gacctcaaac
gcctagctgc caatgacctg cagggctgcg ctgtggccac cggcccttac catcccatct ggaccggcag
ggccaccgat gaggagccgc tggggcttcc caagtgctgc cagccagatg ccgctgacaa ggcctcagta
ctggagcctg gaagaccagc ttcggcaggc aatgcgctga agggacgcgt gccgcccggt gacagcccgc
cgggcaacgg ctctggccca cggcacatca atgactcacc ctttgggact ctgcctggct ctgctgagcc
cccgctcact gcagtgcggc ccgagggctc cgagccacca gggttcccca cctcgggccc tcgccggagg
ccaggctgtt cacgcaagaa ccgcacccgc agccactgcc gtctgggcca ggcaggcagc gggggtggcg
ggactggtga ctcagaaggc tcaggtgccc tacccagcct cacctgcagc ctcacccccc tgggcctggc
gctggtgctg tggacagtgc ttgggccctg ctgaccccca g

Vectors

Vectors comprising nucleic acids encoding the polypeptides or fragments thereof of the present invention may also be used to produce polypeptide for use in the methods of the invention. The choice of vector and expression control sequences to which such nucleic acids are operably linked depends on the functional properties desired, e.g., protein expression, and the host cell to be transformed.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status, or a change in temperature, in the host cell medium.

The vector can include a prokaryotic replicon, i.e., a DNA sequence having the ability to direct autonomous replication and maintenance of the recombinant DNA molecule extra-chromosomally in a bacterial host cell. Such replicons are well known in the art. In addition, vectors that include a prokaryotic replicon may also include a gene whose expression confers a detectable marker such as a drug resistance. Examples of bacterial drug-resistance genes are those that confer resistance to ampicillin or tetracycline.

Vectors that include a prokaryotic replicon can also include a prokaryotic or bacteriophage promoter for directing expression of the coding gene sequences in a bacterial host cell. Promoter sequences compatible with bacterial hosts are typically provided in plasmid vectors containing convenient restriction sites for insertion of a DNA segment to be expressed. Examples of such plasmid vectors are pUC8, pUC9, pBR322 and pBR329 (BioRad Laboratories, Hercules, Calif.), pPL and pKK223. Any suitable prokaryotic host can be used to express a recombinant DNA molecule encoding a protein used in the methods of the invention.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. The neomycin phosphotransferase (neo) gene is an example of a selectable marker gene (Southern et al., J. Mol. Anal. Gezet. 1:327-341 (1982)). Additional elements may also be needed for optimal synthesis of mRNA. These elements may include signal sequences, splice signals, as well as transcriptional promoters, enhancers, and termination signals.

In one embodiment, a proprietary expression vector of Biogen IDEC, Inc., referred to as NEOSPLA (U.S. Pat. No. 6,159,730) may be used. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression upon transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). Additional eukaryotic cell expression vectors are known in the art and are commercially available. Typically, such vectors contain convenient restriction sites for insertion of the desired DNA segment. Exemplary vectors include pSVL and pKSV-10 (Pharmacia), pBPV-1, pml2d (International Biotechnologies), pTDT1 (ATCC 31255), retroviral expression vector pMIG and pLL3.7, adenovirus shuttle vector pDC315, and AAV vectors. Other exemplary vector systems are disclosed e.g., in U.S. Pat. No. 6,413,777.

In general, screening large numbers of transformed cells for those which express suitably high levels of the antagonist is routine experimentation which can be carried out, for example, by robotic systems.

Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (Adm1P)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615.

The recombinant expression vectors may carry sequences that regulate replication of the vector in host cells (e.g., origins of replication) and selectable marker genes. The selectable marker gene facilitates selection of host cells into which the vector has been introduced (see, e.g., Axel, U.S. Pat. Nos. 4,399,216; 4,634,665 and 5,179,017). For example, typically the selectable marker gene confers resistance to a drug, such as G418, hygromycin or methotrexate, on a host cell into which the vector has been introduced. Frequently used selectable marker genes include the dihydrofolate reductase (DHFR) gene (for use in dhfr-host cells with methotrexate selection/amplification) and the neo gene (for G418 selection).

Vectors encoding polypeptides or polypeptide fragments can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al, Proc. Natl. Acad. Sci. USA 69:2110-14 (1972)). For transformation of vertebrate cells, electroporation, cationic lipid or salt treatment methods can be employed. See, e.g., Graham et al., Virology 52:456-467 (1973); Wigler et al., Proc. Natl. Acad. Sci. USA 76:1373-76 (1979).

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to NSO, SP2 cells, baby hamster kidney (BHK) cells, monkey kidney cells (COS), human hepatocellular carcinoma cells (e.g., Hep G2), A549 cells DG44 and DUXB11 (Chinese Hamster Ovary lines, DHFR minus), HELA (human cervical carcinoma), CV1 (monkey kidney line), COS (a derivative of CV1 with SV40 T antigen), R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

Expression of polypeptides from production cell lines can be enhanced using known techniques. For example, the glutamine synthetase (GS) system is commonly used for enhancing expression under certain conditions. See, e.g., European Patent Nos. 0 216 846, 0 256 055, and 0 323 997 and European Patent Application No. 89303964.4.

Eukaryotic cell expression vectors are known in the art and are commercially available. Typically, such vectors contain convenient restriction sites for insertion of the desired DNA segment. Exemplary vectors include pSVL and pKSV-10, pBPV-1, pml2d, pTDT1 (ATCC 31255), retroviral expression vector pMIG, adenovirus shuttle vector pDC315, and AAV vectors.

Eukaryotic cell expression vectors may include a selectable marker, e.g., a drug resistance gene. The neomycin phosphotransferase (neo) gene is an example of such a gene (Southern et al., J. Mol. Anal. Genet. 1:327-341 (1982)).

Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (Adm1P)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615.

The recombinant expression vectors may carry sequences that regulate replication of the vector in host cells (e.g., origins of replication) and selectable marker genes. The selectable marker gene facilitates selection of host cells into which the vector has been introduced (see, e.g., Axel, U.S. Pat. Nos. 4,399,216; 4,634,665 and 5,179,017). For example, typically the selectable marker gene confers resistance to a drug, such as G418, hygromycin or methotrexate, on a host cell into which the vector has been introduced. Frequently used selectable marker genes include the dihydrofolate reductase (DHFR) gene (for use in dhfr-host cells with methotrexate selection/amplification) and the neo gene (for G418 selection).

Nucleic acid molecules encoding the polypeptides or fragments thereof of the present invention, and vectors comprising these nucleic acid molecules, can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., Proc. Natl. Acad. Sci. USA 69:2110-14 (1972)). For transformation of vertebrate cells, electroporation, cationic lipid or salt treatment methods can be employed. See, e.g., Graham et al., Virology 52:456-467 (1973); Wigler et al., Proc. Natl. Acad. Sci. USA 76:1373-76 (1979).

Host Cells

Host cells for expression of a polypeptide or fragment thereof of the present invention for use in a method of the invention may be prokaryotic or eukaryotic. Exemplary eukaryotic host cells include, but are not limited to, yeast and mammalian cells, e.g., Chinese hamster ovary (CHO) cells (ATCC Accession No. CCL61), NIH Swiss mouse embryo cells N1H-3T3 (ATCC Accession No. CRL1658), and baby hamster kidney cells (BHK). Other useful eukaryotic host cells include insect cells and plant cells. Exemplary prokaryotic host cells are E. coli and Streptomyces.

Mammalian cell lines available as hosts for expression are known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines.

Expression of polypeptides from production cell lines can be enhanced using known techniques. For example, the glutamine synthetase (GS) system is commonly used for enhancing expression under certain conditions. See, e.g., European Patent Nos. 0 216 846, 0 256 055, and 0 323 997 and European Patent Application No. 89303964.4.

Pharmaceutical Compositions

The polypeptides, polypeptide fragments, polynucleotides, vectors and host cells of the present invention may be formulated into pharmaceutical compositions for administration to mammals, including humans. The pharmaceutical compositions used in the methods of this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions used in the methods of the present invention may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In the methods of the invention, the polypeptides or fragments thereof of the present invention are administered in such a way that they cross the blood-brain barrier. This crossing can result from the physico-chemical properties inherent in the polypeptide molecule itself, from other components in a pharmaceutical formulation, or from the use of a mechanical device such as a needle, cannula or surgical instruments to breach the blood-brain barrier. Where the polypeptide or fragment thereof of the present invention is a molecule that does not inherently cross the blood-brain barrier, e.g., a fusion to a moiety that facilitates the crossing, suitable routes of administration are, e.g., intrathecal or intracranial. Where the polypeptide or fragment thereof of the present invention is a molecule that inherently crosses the blood-brain barrier, the route of administration may be by one or more of the various routes described below.

Sterile injectable forms of the compositions used in the methods of this invention may be aqueous or oleaginous suspension. These suspensions may be formulated according to techniques known in the art using suitable dispersing or wetting agents and suspending agents. The sterile, injectable preparation may also be a sterile, injectable solution or suspension in a non-toxic parenterally acceptable diluent or solvent, for example as a suspension in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered at specific fixed or variable intervals, e.g., once a day, or on an “as needed” basis.

Certain pharmaceutical compositions used in the methods of this invention may be orally administered in an acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

The amount of a polypeptide or fragment thereof of the present invention that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response).

The methods of the invention use a “therapeutically effective amount” or a “prophylactically effective amount” of a polypeptide or fragment thereof of the present invention. Such a therapeutically or prophylactically effective amount may vary according to factors such as the disease state, age, sex, and weight of the individual. A therapeutically or prophylactically effective amount is also one in which any toxic or detrimental effects are outweighed by the therapeutically beneficial effects.

A specific dosage and treatment regimen for any particular patient will depend upon a variety of factors, including the particular polypeptide or fragment thereof of the present invention used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

In the methods of the invention the polypeptides or fragments thereof of the present invention are generally administered directly to the nervous system, intracerebroventricularly, or intrathecally. Compositions for administration according to the methods of the invention can be formulated so that a dosage of 0.001-10 mg/kg body weight per day of the polypeptide or fragment thereof of the present invention is administered. In some embodiments of the invention, the dosage is 0.01-1.0 mg/kg body weight per day. In some embodiments, the dosage is 0.001-0.5 mg/kg body weight per day.

Supplementary active compounds also can be incorporated into the compositions used in the methods of the invention. For example, a polypeptide or fragment thereof of the present invention, or a fusion protein thereof, may be coformulated with and/or coadministered with one or more additional therapeutic agents, thereby acting as a drug delivery targeting agent.

For treatment with a polypeptide or fragment thereof of the present invention, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly.

In some methods, two or more polypeptides or fragments thereof of the present invention are administered simultaneously, in which case the dosage of each polypeptide administered falls within the ranges indicated. Supplementary active compounds also can be incorporated into the compositions used in the methods of the invention. For example, an antibody may be coformulated with and/or coadministered with one or more additional therapeutic agents.

The invention encompasses any suitable delivery method for a polypeptide or fragment thereof of the present invention to a selected target tissue, including bolus injection of an aqueous solution or implantation of a controlled-release system. Use of a controlled-release implant reduces the need for repeat injections.

The polypeptides or fragments thereof of the present invention used in the methods of the invention may be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., “High Activity Iodine-125 Interstitial Implant For Gliomas,” Int. J Radiation Oncology Biol. Phys. 24(4):583-91 (1992); Gaspar et al, “Permanent 125I Implants for Recurrent Malignant Gliomas,” Int. J. Radiation Oncology Biol. Phys. 43(5):977-82 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., “The Safety of Interstitial Chemotherapy with BCNU-Loaded Polymer Followed by Radiation Therapy in the Treatment of Newly Diagnosed Malignant Gliomas: Phase I Trial,” J. Neuro-Oncology 26:111-23 (1995).

The compositions may also comprise a polypeptide or fragment thereof of the present invention dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J Biomed. Mater. Res. 15:167-277 (1981); Langer, Chem. Tech. 12:98-105 (1982)) or poly-D-(−)-3hydroxybutyric acid (EP 133,988).

In some embodiments, a polypeptide or fragment thereof of the present invention is administered to a patient by direct infusion into an appropriate region of the brain. See, e.g., Gill et al., “Direct brain infusion of glial cell line-derived neurotrophic factor in Parkinson disease,” Nature Med. 9: 589-95 (2003). Alternative techniques are available and may be applied to administer a polypeptide or fragment thereof according to the present invention. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

Treatment Methods

One embodiment of the present invention provides methods for treating a disease, disorder or injury associated with hyper or hypo activity of neurons, abnormal neuron sprouting and/or neurite outgrowth, e.g., scizophrenia in an animal suffering from such disease, the method comprising, consisting essentially of, or consisting of administering to the animal an effective amount of a Nogo fragment of the present invention.

Additionally, the invention is directed to a method for enhancing neurite outgrowth inhibition in a mammal comprising, consisting essentially of, or consisting of administering a therapeutically effective amount of a Nogo polypeptide fragment of the present invention.

Also included in the present invention is a method of enhancing neurite outgrowth inhibition, comprising, consisting essentially of, or consisting of contacting a neuron with an effective amount of a polypeptide or fragment thereof of the present invention as described above.

A Nogo polypeptide fragment of the present invention can be prepared and used as a therapeutic agent that enhances the ability to negatively regulate neuronal growth or regeneration.

Diseases or disorders which may be treated or ameliorated by the methods of the present invention include diseases, disorders or injuries which relate to the hyper- or hypo-activity of neurons, abnormal neuron sprouting, and/or abnormal neurite outgrowth. Such disease include, but are not limited to, schizophrenia, bipolar disorder, obsessive-compulsive disorder (OCD), Attention Deficit Hyperactivity Disorder (ADHD), Downs Syndrome, and Alzheimer's disease.

In Vitro Methods

The present invention also includes methods of enhancing neuronal cell growth inhibition in vitro. For example, the invention includes in vitro methods for inhibiting abnormal neuronal cell growth, inhibiting neurite outgrowth, or inhibiting abnormal neuron sprouting.

Targeting and Screening Assays

The present invention also includes methods of screening for drug candidates using the polypeptides or fragments thereof of the present invention. For example, the polypeptides or fragment thereof of the present invention could be used to screen for small molecules that bind to NgR. In addition, the polypeptides or fragment thereof of the present invention could be used as a drug delivery targeting agent to target neurons or cells that specifically express NgR.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Amino Nogo Fragments Bind to NgR

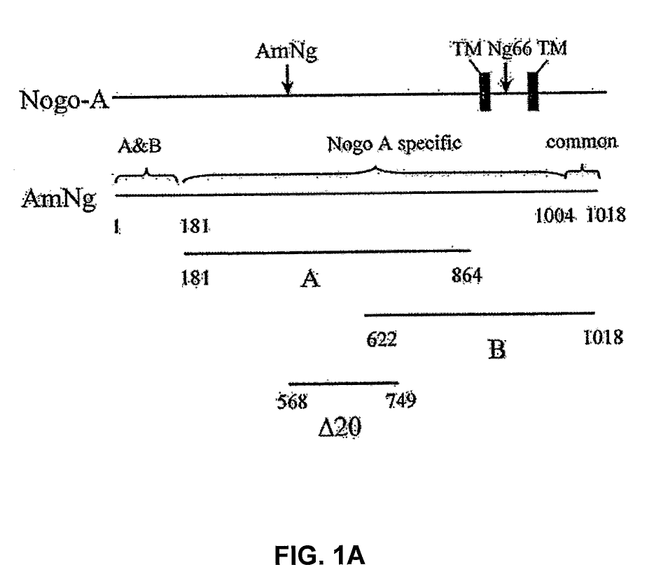

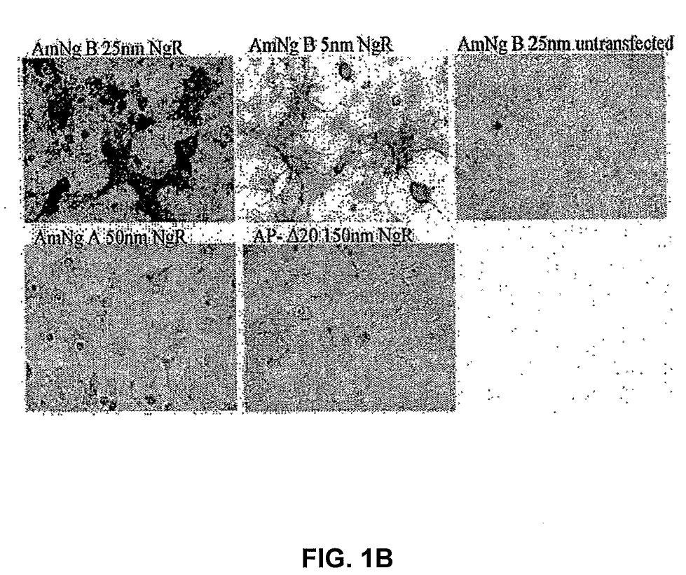

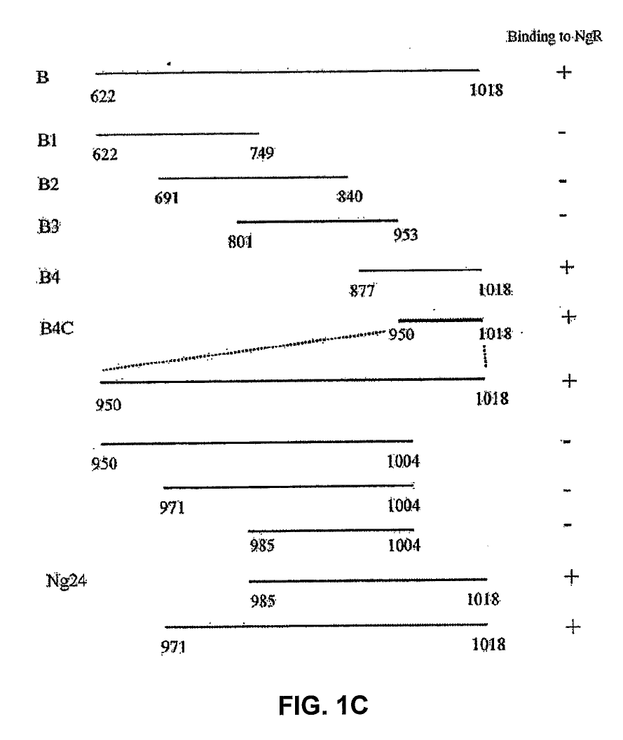

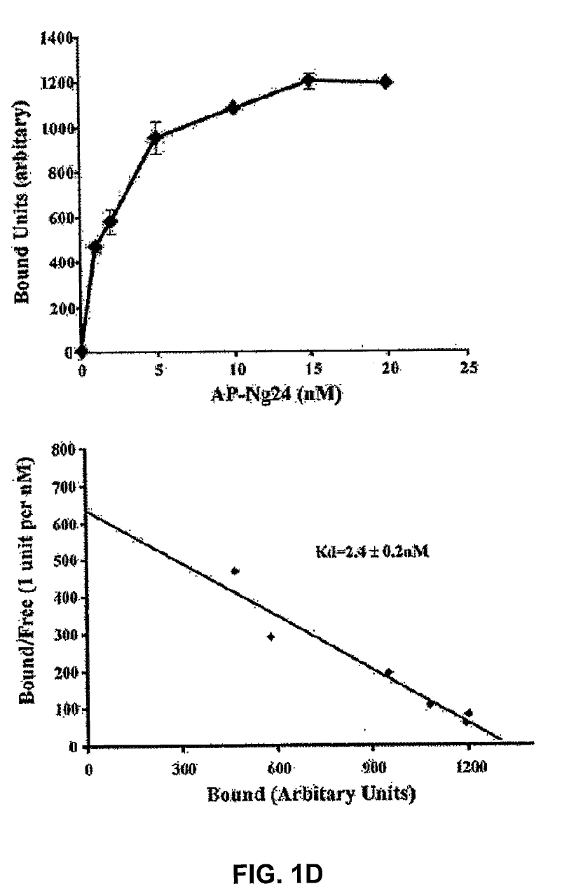

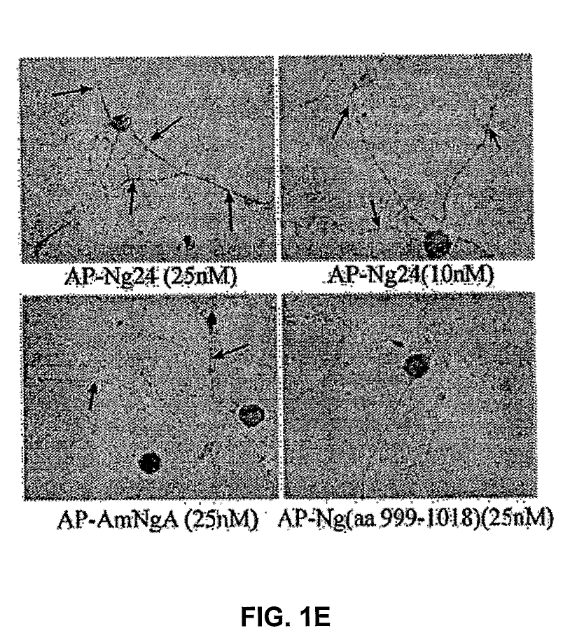

This example demonstrates that the carboxyl terminus of the Amino-Nogo domain interacts with NgR with high affinity. Several alkaline phosphatase (AP) fusion proteins containing various Nogo-A segments derived from regions between the amino terminus and the first hydrophobic segment were examined to identify the mechanism of Amino-Nogo-A action. To generate additional AP fusion proteins, human amino Nogo fragments were amplified and ligated to the pcAP6 vector digested with restriction enzymes EcoRI and XhoI as described (Fournier, A. E., et al., Nature 409:341-346 (2001)). Plasmids were then transfected into HEK293T cells and conditioned media were collected after 7 days. None of these fragments bind with high affinity to non-transfected COS-7 cells. While examining presumed control conditions, we unexpectedly observed that the carboxyl half of Amino-Nogo (fragment B) exhibited high affinity binding to COS-7 cells expressing NgR (FIG. 1B). This binding is saturable with a Kd indistinguishable from that for AP-Nogo-66 association with NgR (Table I). To better define the region responsible for Amino-Nogo interaction with NgR, a range of truncation mutants of Amino-Nogo were examined as AP fusion proteins. Subdivision of the B fragment into overlapping 150 aa segments reveals that the NgR interaction site is localized to the most carboxyl terminal segment. In fact, the NgR-interacting segment of Amino-Nogo is fully accounted for in the extreme carboxyl 24 amino acids (aa 995-1018, Amino-Nogo-A-24) (Table I and FIG. 1D). The Ile residue located at aa 995 is important for high affinity binding, as are the next carboxyl 18 aa from residue 996 to residue 1013 (Table I). We named this domain (aa 995-1013) as Amino-Nogo-A-19.

The 19 aa NgR-binding residues of Amino-Nogo-A are encoded by nucleotides that span the splice site (aa 1004/1005) between the Nogo-A specific exon of the nogo gene and the 5′ common exon of the gene (Chen, M. S., et al., Nature 403:434-439 (2000); GrandPre, T., et al., Nature 403:439-444 (2000); Oertle, T., et al., J. Mol. Biol. 325:299-323 (2003a)). AP fusion proteins comprised of aa from the Nogo-A-specific region alone do not bind to NgR (aa 950-1004). Amino-Nogo residues of Nogo-B or Nogo-C also fail to associate with NgR-expressing cells (Table I). Thus, this second high affinity NgR interacting domain is Nogo-A-specific, and is immediately amino terminal to the hydrophobic segment that separates it from Nogo-66.

If these Amino-Nogo fragments are to play a role in regulating neurite outgrowth then they would be expected to bind to neuronal processes. Previously, we have shown that AP-Nogo-66 binds to NgR on DRG processes (Fournier, A. E., et al., Nature 409:341-346 (2001)). As expected from COS-7 NgR binding experiments, the carboxyl terminal 24 aa of Amino-Nogo can also mediate AP fusion protein binding to DRG axons but a shorter fragment (aa 999-1018) of Nogo-A fails to interact with DRG neurons (FIG. 1E). The amino terminal A fragment of Amino-Nogo also binds to DRG axons, presumably through NgR-independent mechanisms.

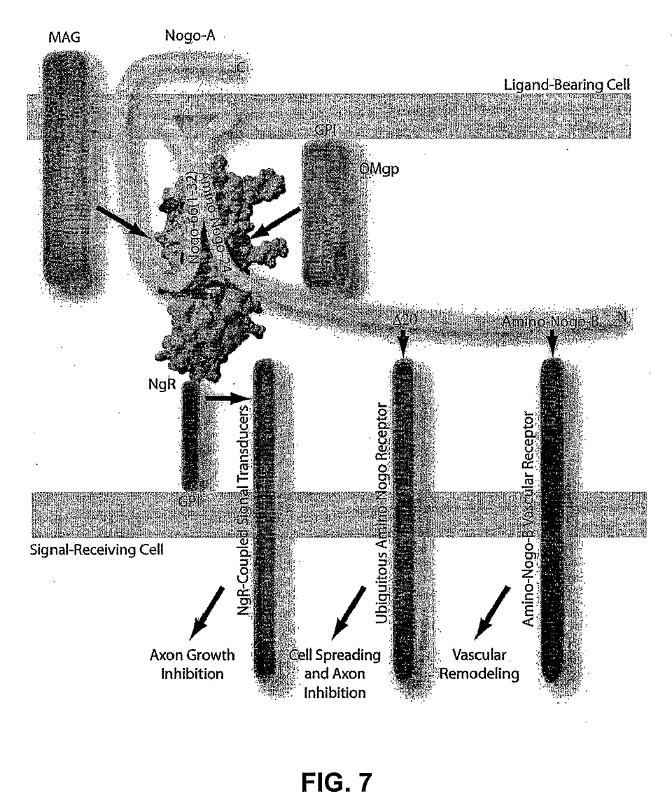

It has been reported that a fraction of Nogo-A in oligodendrocytes is situated in a conformation exposing both the amino terminus and the Nogo-66 domain at the cell surface (Oertle, T., et al., J. Neurosci. 23:5393-5406 (2003b)). The more amino terminal hydrophobic segment of Nogo-A is proposed to insert into the plasma membrane as a loop. While not being bound by theory, this conformation is predicted to bring the Amino-Nogo-A-19 segment and the Nogo-66 domain at the cell surface into close proximity at the cell surface (FIG. 7). The ability of both of these domains to interact with NgR is consistent with a physiological role for this conformation.

TABLE I
Binding affinity of Amino Nogo Fragments to NgR

| Amino Acid Number | Amino Acid Sequence | NgR Kd (nM) |
|---|---|---|
| a.a. 181-864 (AmNg A) | | No Binding at |
| | | 150 nM |
| a.a. 622-1018 (AmNg B) | | 6.66 ± 1.49 |
| a.a. 877-1018 (AmNg B4) | | 9.01 ± 6.36 |
| a.a. 950-1018 (AmNg B4C) | | 3.51 ± 3.36 |
| a.a. 971-1018 | | 2.69 ± 1.32 |
| a.a. 995-1018 | IFSAELSKTSVVDLLYWRDIKKTG | 2.43 ± 0.51 |
| (Amino-Nogo-A-24) | | |
| a.a. 995-1015 | IFSAELSKTSVVDLLYWRDIK | 4.55 ± 3.66 |
| a.a. 995-1014 | IFSAELSKTSVVDLLYWRDI | 3.19 ± 0.12 |
| a.a. 995-1013 | IFSAELSKTSVVDLLYWRD | 2.48 ± 0.72 |
| (Amino-Nogo-A-19) | | |
| a.a. 996-1018 | FSAELSKTSVVDLLYWRDIKKTG | 26.59 ± 6.86 |
| a.a. 1000-1018 | LSKTSVVDLLYWRDIKKTG | No binding at |
| | | 25 nM |
| a.a. 1005-1018 | VVDLLYWRDIKKTG | No binding at |
| | | 25 nM |
| a.a. 950-1004 | ............IFSAELSKTS | No binding at |
| | | 50 nM |
| Amino of NgC | MDGQKKNWKDKVVDLLYWRDIKKTG | No binding at |
| | | 25 nM |
| Amino of NgB | | No binding at |
| | | 100 nM |

Binding Kds for AP fused Amino-Nogo fragments were measured by applying conditioned media containing AP fusion protein to NgR expressing COS-7 cells. Bound AP was stained and measured.

### Example 2

### Inhibition of Cell Spreading and Axon Outgrowth by Amino Nogo is Separable from NgR Binding

It has been recognized that the Amino-Nogo-A protein inhibits non-neuronal cell spreading and axonal outgrowth when the protein is substrate bound (Chen, M. S., et al., Nature 403:434-439 (2000); Fournier, A. E., et al., Nature 409:341-346 (2001)). Work by Oertle et al. has suggested that specific aa stretches near the amino terminus and the middle of Amino-Nogo-A are responsible for this activity (Oertle, T., et al., J. Neurosci. 23:5393-5406 (2003b)). The later domain has been termed Δ20. To determine whether the NgR-interacting aa of Amino-Nogo-A described in Example 1 regulate cell spreading and axonal outgrowth, various fragments were expressed as GST fusion proteins and purified from E. coli. To generate GST fusion proteins, amino Nogo fragments were cloned in pGEX2T (Amersham Pharmacia). Native and soluble GST fusion proteins were expressed and purified as described (GrandPre, T., et al., Nature 403:439-444 (2000)). COS-7 binding assays were done as described (Fournier, A. E., et al., Nature 409:341-346 (2001)). Bound AP to COS-7 cells was measured using NIH image software. Fibroblast spreading and cDRG outgrowth assay were done as described (Fournier, A. E., et al., Nature 409:341-346 (2001)) with some modifications. Briefly, 50 μl of purified GST fusion protein or peptides diluted in PBS was pipetted into polylysine precoated 96 well plates (Becton Dickson Biocoat plates) and dried overnight at room temperature. For fibroblast spreading assay, subconfluent COS-7 cells were then plated for 1 hour in serum containing medium before fixation and staining with rho damine-phalloidin.

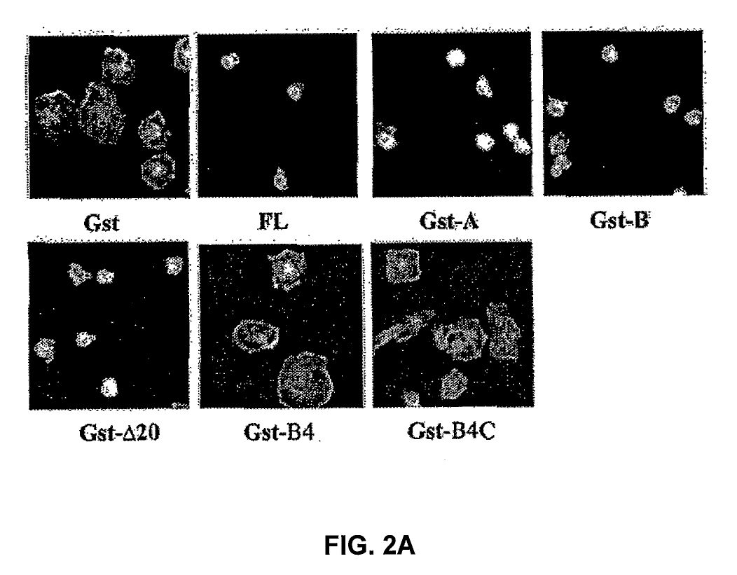

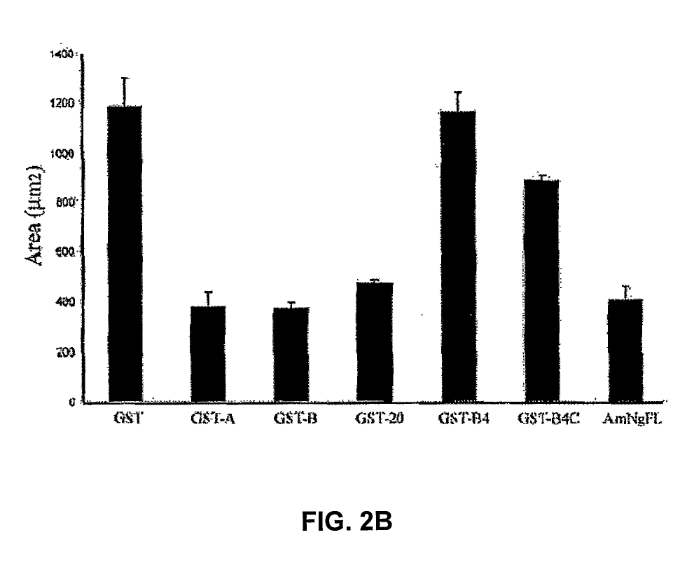

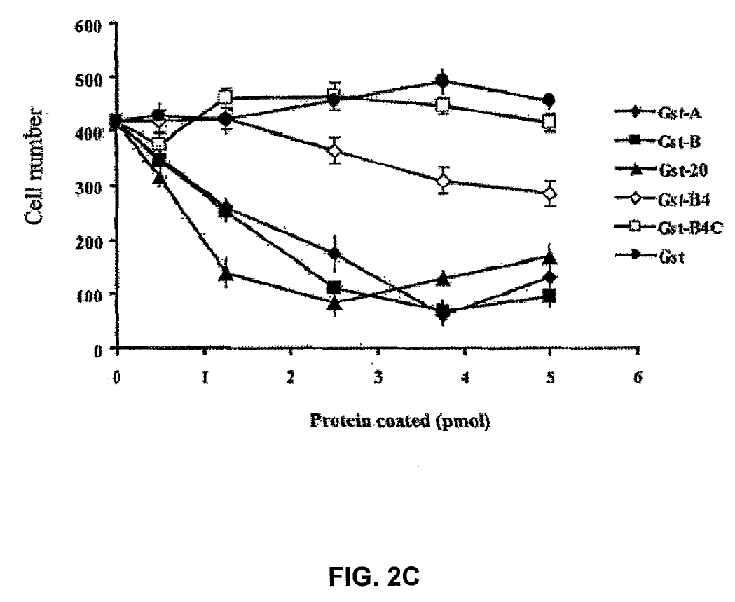

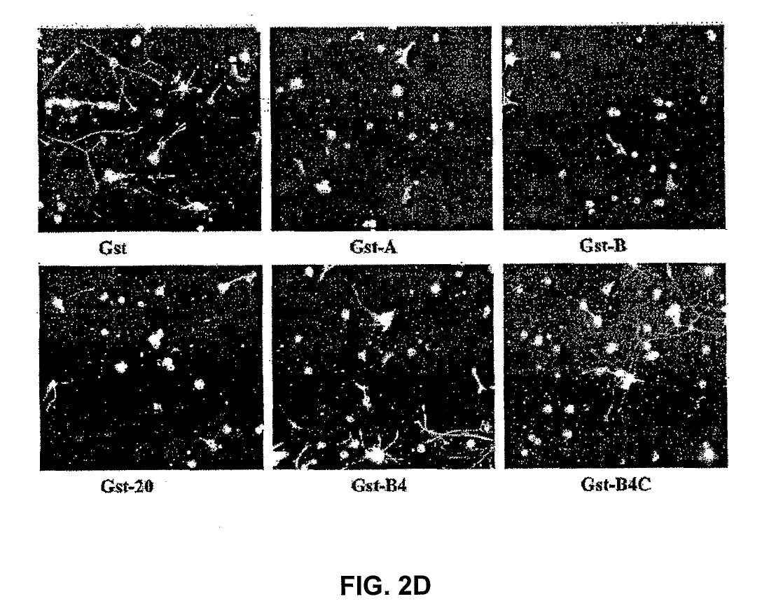

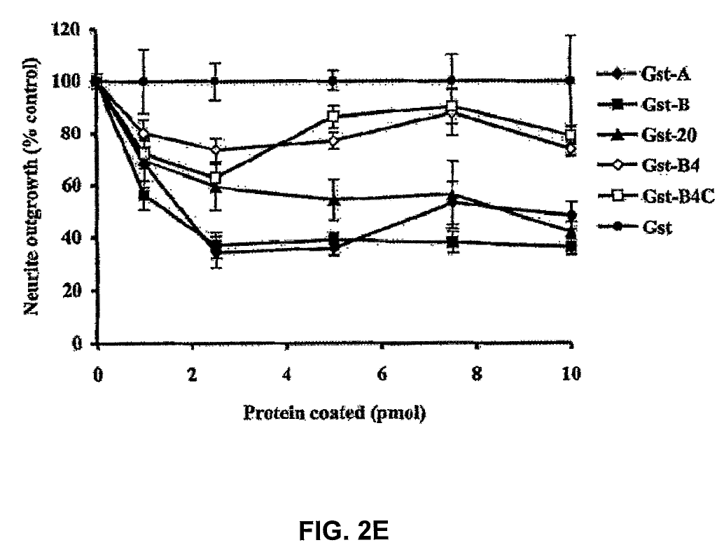

Fragments containing portions of the Δ20 region significantly reduce COS-7 cell attachment and spreading (FIG. 2A-C). The entire Δ20 region does not appear essential for regulation of COS-7 cells since the B fragment of Amino-Nogo is active but contains only a portion of the Δ20 region. Fragments consisting of the carboxyl terminal 75 aa (B4C) or 150 aa (B4) lack the Δ20 region but possess the entire 19 aa NgR binding region (FIG. 1C). The B4 and B4C proteins do not alter COS-7 morphology when presented as a substrate (FIG. 2A-C). Thus, inhibition of fibroblast spreading is separable from NgR binding by Amino-Nogo-A.

The same GST-Amino-Nogo proteins were tested for their ability to reduce neurite outgrowth from chick E13 DRG neurons. For cDRG outgrowth assay, dissociated E13 cDRG neurons were plated for 6 hours before fixation. Neurons were stained with anti-Neurofilament (Sigma Catalog #N4142) and anti-HuC/D (Molecular Probes A-21271) antibodies. Cell area, number of attached cells and neurite length were measured using the Imageexpress machine and software (Axon Instrument).

As shown previously for the entire Amino-Nogo domain, those subfragments containing portions of the Δ20 region are inhibitory for neurite outgrowth (Fournier, A. E., et al., Nature 409:341-346 (2001); Oertle, T., et al., J. Neurosci. 23:5393-5406 (2003b)) (FIGS. 2D and 2E). Since these cultures are known to express NgR and respond to binding with Nogo-66, we tested whether the NgR-binding B4 and B4C fragments of Amino-Nogo would alter neurite outgrowth. Unexpectedly, substrates coated with the NgR-binding B4 and B4C fragments of Amino-Nogo-A were not inhibitory for axonal growth (FIGS. 2D and 2E). Thus, the NgR-binding domain of Amino-Nogo does not bind to NgR-negative COS-7 cells and when bound to NgR-positive neurons it does not alter axon growth. Given that the NgR-binding domain of 19 aa (Amino-Nogo-A-19) does not alter cell spreading or axonal outgrowth, explains why it was not detected in initial assays. This domain is present only in Nogo-A, providing one basis for Nogo-A being a more potent inhibitor of axonal growth than Nogo-C (Chen, M. S., et al., Nature 403:434-439 (2000); GrandPre, T., et al., Nature 403:439-444 (2000)).

In addition, we and others have previously documented that substrate bound or aggregated Amino-Nogo inhibited fibroblast spreading and neurite outgrowth (Chen, M. S., et al., Nature 403:434-439 (2000); Fournier, A. E., et al., Nature 409:341-346 (2001); Oertle, T., et al., J. Neurosci. 23:5393-5406 (2003b)). As suggested by these properties, we confirm that the Amino-Nogo domain responsible for these activities does not bind to NgR. The molecular basis for these actions remains unknown. At least a significant portion of this activity can be localized to a Δ20 segment near the middle of Amino-Nogo. The amino terminus of Nogo has recently been recognized to have another NgR independent action via an extreme amino terminal domain that is shared between Nogo-A and Nogo-B. This domain has a selective role in remodeling the vasculature after injury (Acevedo, L., et al., Nat Med, 10:382-388 (2004)). Thus, Nogo appears to have multiple functional domains and receptors. The Δ20 region of Nogo-A does not bind to NgR but is non-permissive as a substrate for multiple cell types. The amino terminal segment of Nogo-A and Nogo-B has no affinity for NgR, but does regulate vascular endothelial and smooth muscle cell migration through an unidentified receptor.

### Example 3

### Carboxyl Region of Amino-Nogo-A Binds to the LRR Domain of NgR

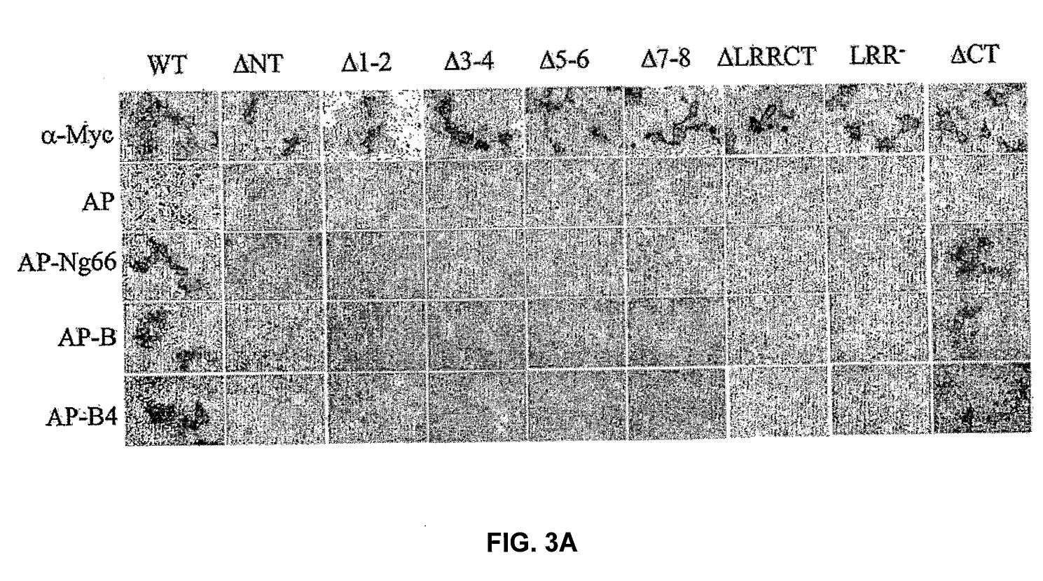

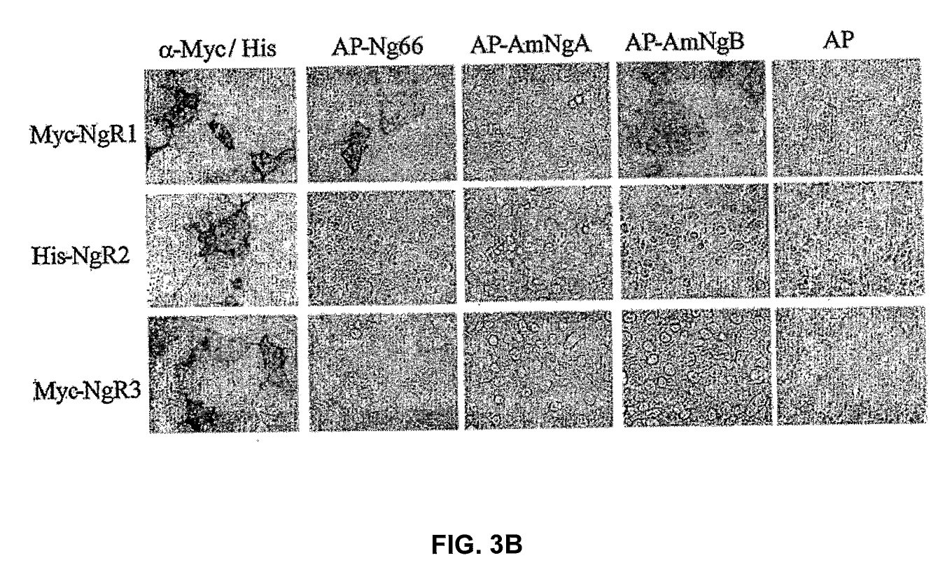

Since Amino-Nogo binding to neuronal NgR does not inhibit axon outgrowth, we sought to determine whether the specificity of Amino-Nogo for NgR was similar to that of the Nogo-66 domain. As for Nogo-66, MAG and OMgp (Barton, W. A., et al., Embo J. 22:3291-3302 (2003); Founier, A. E., et al., Nature 409:341-346 (2001); Wang, K. C., et al., Nature 417:941-944 (2002b)), deletions of any two LRRs eliminated binding to NgR for the Amino-Nogo-B4C fragment (FIG. 3A). Similarly, the cysteine rich LRR-NT and LRR-CT capping domains are essential for Amino-Nogo-B4C binding. In contrast, deletion of the unique signaling domain of NgR extending from the LRR region to the GPI anchorage site (CT domain) did not alter Amino-Nogo-B4C binding. NgR is part of gene family that includes NgR2 and NgR3. When expressed on the surface of COS-7 cells, these related proteins do not bind AP-Nogo-66 or AP-MAG or AP-OMgp (Barton, W. A., et al., Embo J. 22:3291-3302 (2003)). Similarly, NgR2 and NgR3 are not binding partners for Amino-Nogo (FIG. 3B). By these measures, the NgR requirements for Nogo-66 and Amino-Nogo-B4C binding are indistinguishable.

### Example 4

### NgR Residues Required for the Binding of Different Ligands

NgR has the capacity to bind Nogo-66, MAG, OMgp, and Lingo-1 plus Amino-Nogo. Previous work had been contradictory as to whether binding sites for Nogo-66 and MAG were separate or overlapping. Using NEP1-40 antagonist of Nogo-66, we did not observe inhibition of MAG interactions with NgR (Liu, B. P., et al., Science 297:1190-1193 (2002)). With a sterically encumbered AP-Nogo-66 ligand, some competition with MAG-Fc binding to NgR was detected (Domeniconi, M., et al., Neuron 35:283-290 (2002)). Since the structure of the NgR is now defined (Barton, W. A., et al, Embo J 22:3291-3302 (2003); Domeniconi, M., et al., Neuron 35:283-290 (2002); He, X. L., et al., Neuron 38:177-185 (2003)), we probed its surface for ligand binding sites by Ala substitutions.

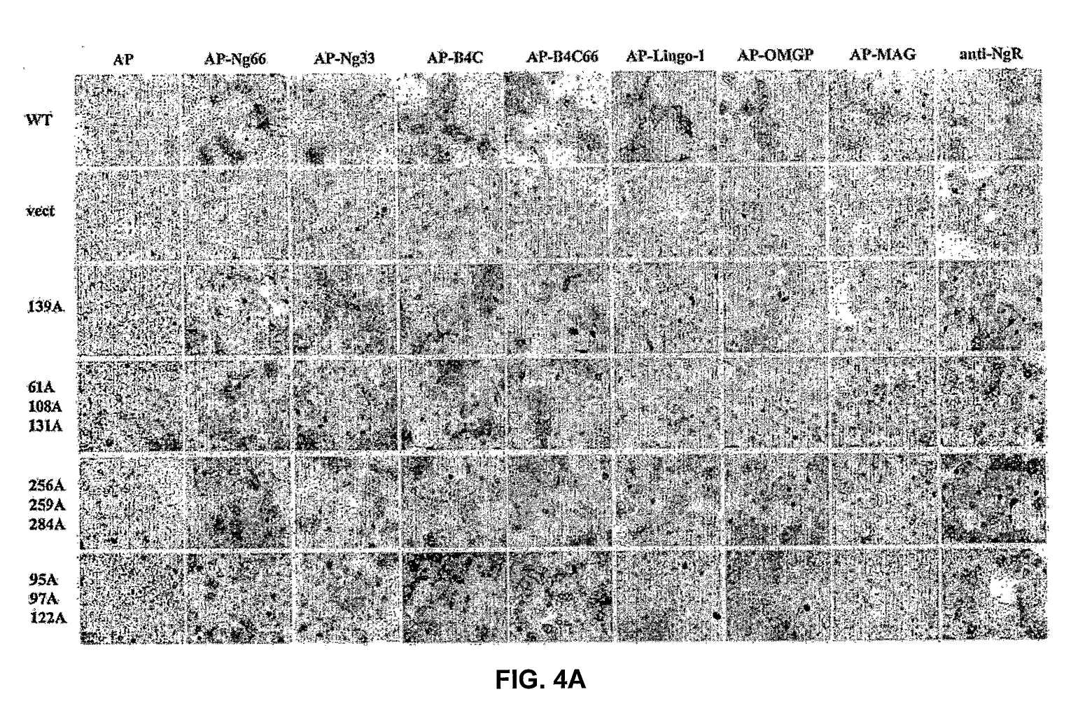

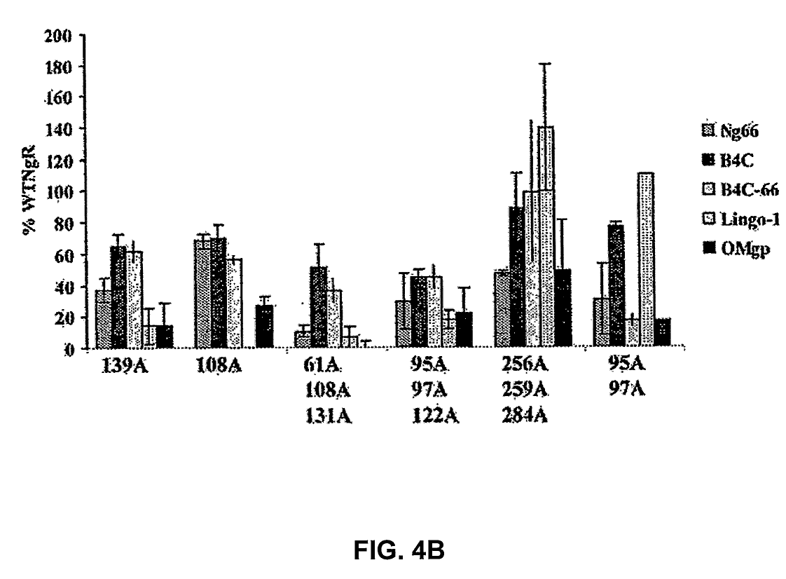

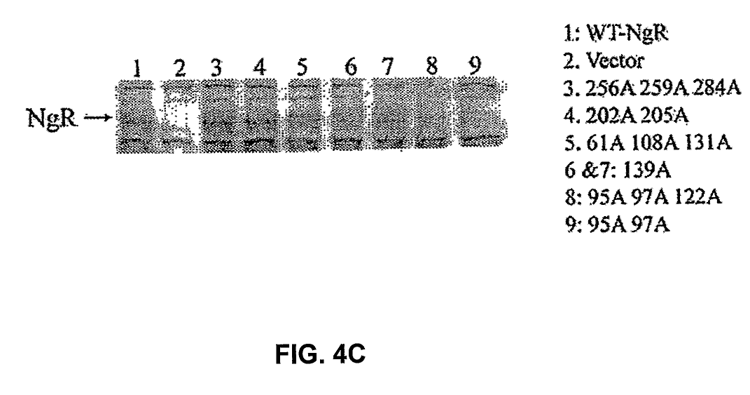

To better define how multiple ligands bind to the NgR protein, we examined a series of Ala-substituted NgR for ligand binding activity. NgR mutagenesis was done using the Quick Change Multisite Directed Mutagenesis Kit (Stratagene catalog #200514). Human NgR1 was used as a template. Ala substitutions were generated for each of the charged residues predicted to be solvent accessible at the surface of the ligand binding domain of NgR (Barton, W. A., et al., Embo J. 22:3291-3302 (2003); He, X. L., et al., Neuron 38:177-185 (2003)). We generated mutants in which 1-8 surface residues localized within 5 A of one another were Ala-substituted. Because of the coiling nature of the LRR structure, as residues juxtaposed on the protein surface are commonly separated by approximately 25 residues in the primary structure. In addition to mutations in specific charged surface patches, other mutations were targeted to glycosylation sites and to regions predicted to be involved in ligand binding based on the NgR structure (Barton, W. A., et al., Embo J. 22:3291-3302 (2003); He, X. L., et al., Neuron 38:177-185 (2003)). In addition, a variant corresponding to a human polymorphism was examined (D259N). None of the mutations altered the Leu residues that define the LRR structure itself or the Cys residues critical in the amino and carboxyl terminal capping domains. The vast majority of such Ala surface substitution mutants were expressed as immunoreactive polypeptides with a molecular weight and an expression level indistinguishable from wild type NgR (FIG. 4C and data not shown). Those that did not were excluded from further analysis. Moreover, all of the mutant NgR that were analyzed for ligand binding exhibited a cellular distribution in transfected COS-7 identical to that of the wild type protein. Notably, those mutations that removed the glycosylation sites in the aa 27-310 region did not alter expression levels of surface expression, although molecular weight was reduced by immunoblot analysis (data not shown).

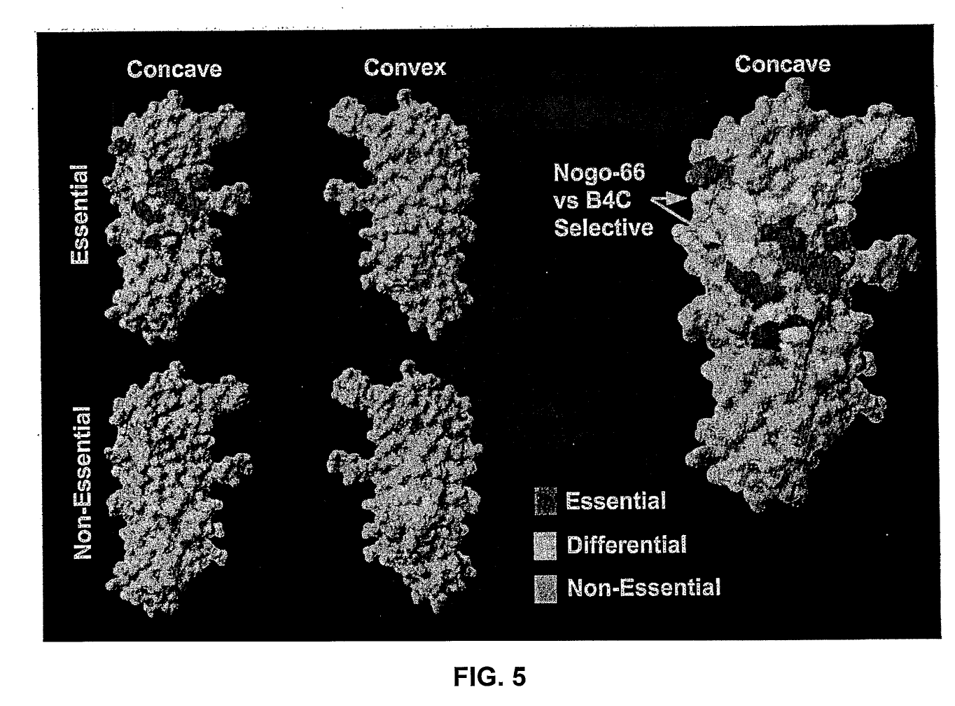

This collection of 74 individual NgR mutants was interrogated for AP-Nogo-66, AP-Amino-Nogo-B4C, AP-MAG, AP-OMgp, and AP-Lingo-1 binding. AP-Nogo66, AP-MAG, AP-OMGP and AP-Lingo-1 constructs are described elsewhere (Fournier, A. E., et al., Nature 409:341-346 (2001); Liu, B. P., et al., Science 297:1190-1193 (2002); Mi, S., et al., Nat. Neurosci. 7:221-228 (2004); Wang, K. C., et al., Nature 417:941-944 (2002b)). The properties of the NgR mutants fell into one of three major categories (Table II and FIG. 5). A number of Ala substituted NgR polypeptides bound all of the ligands at wild type levels. We conclude that the corresponding aa do not play an essential role in ligand interactions. Many of these residues are situated on the convex “outside” of the NgR structure, indicating that this surface is not a primary site for intermolecular interactions. In addition, a significant extent of the concave surface is dispensable for ligand binding.

A second group of mutants exhibited weak or no binding for each of the ligands. While not being bound by theory, one interpretation is that these residues are required for NgR folding, so that their substitution with Ala results in misfolded protein with no ligand binding. However, there are several reasons to favor the alternative hypothesis that many of these residues contribute to the binding of multiple NgR ligands in a common binding pocket.

Critically, the NgR expression levels and subcellular distribution are not altered for these mutants. In contrast, unfolded or misfolded protein might be expected to be unstable and mislocalized. It is also notable that the majority of those residues that camiot be mutated to Ala without a loss of ligand binding are clustered near one another. Thus, we conclude that the NgR surface created by residues including, but not limited to, 67/68, 111/113, 133/136, 158/160, 163, 182/186, and 232/234 constitutes a primary binding site for these ligands. Rat and human NgR are identical at all 13 of these positions. The NgR related proteins, NgR2 and NgR3, each have 10 identical residues, 2 similar/non-identical residues and 1 dissimilar residue at these positions.

The third group of Ala substituted NgR mutants exhibit selective loss of binding for some ligands but not others (Table III and FIG. 4). The preservation of binding affinity for at least one ligand by each member of this class demonstrates the Ala replacements do not prevent NgR folding and surface expression. Most of the NgR residues responsible for differential ligand binding are situated at the perimeter of the primary binding site described above. Many of these substitutions reduce or eliminate MAG, OMgp and Lingo-1 binding without diminishing binding by Nogo-66 or the B4C fragment of Amino-Nogo-A. While not being bound by theory, the simplest interpretation of this topographic relationship is that MAG, OMgp and Lingo-1 require not only a central ligand binding domain that is partially shared with Nogo-66, but also an adjacent group of aa for high affinity binding. This adjacent region includes, for example, aa 78/81, 87/89, 89/90, 95/97, 108, 119/120, 139, 210, and 256/259. Mouse and human NgR are identical at 11 of these 14 residues and similar at 13 of 14. NgR2 exhibits less conservation at these 14 positions with 8 identical aa, 1 similar/non-identical aa and 5 dissimilar aa. For NgR3 there are 6 identical aa, 4 similar/non-identical aa and 4 dissimilar aa.

Of particular interest are those Ala substitutions at aa 95/97 and 139 that reduce Nogo-66 binding to a greater extent than binding by the Amino-Nogo B4C fragment. These residues lie to the non-glycosylated side of the core binding site on the concave face of NgR. The differential binding of these Ala substituted NgR proteins demonstrates that Nogo-66 and Amino-Nogo interact with partially separable sites on NgR. This finding raises the possibility that both domains of one Nogo-A molecule are capable of interacting with one NgR protein.

This analysis demonstrates both similarities and differences between the residues required for binding different ligands. There appears to be a central binding domain required by Amino-Nogo-A-19, Nogo-66, MAG and OMgp ligands. In addition, different ligands require particular residues surrounding this central site. These findings are consistent with partial but incomplete competition between ligands. Because all ligands require surface residues centered on the mid-portion of the concave face of NgR, their mechanism for activating NgR signaling may be similar. The conversion of the Nogo-66 antagonist NEP1-32 to an agonist by fusion to Amino-Nogo-A-24 raises the possibility that this activation mechanism involves altered valency of receptor aggregates through ligation of this central domain.

Because NgR may be considered a target for the development of axonal regeneration therapeutics (Lee, D. H., et al., Nat. Rev. Drug Discov. 2:872-878 (2003)), the definition of this central binding domain shared by multiple ligands may facilitate the design and/or development of small molecule therapeutics blocking all NgR ligands. Accordingly, the variant NgR1 polypeptides of the present invention may be used in screening assays. In contrast, if each ligand requires completely separate residues for binding with high affinity then the chance of developing blockers of all myelin protein action at NgR with a low molecular weight compound would be significantly less.

Lingo-1 has been reported as a component of a signal transducing NgR complex (Mi, S., et al., Nat. Neuiosci. 7:221-228 (2004)). It is notable here that the residues required for its binding to NgR are very similar to those for the ligands MAG and OMgp. While not being bound by theory, because Lingo-1 in also expressed by oligodendrocytes, the binding analysis suggests that it might act as a ligand. Alternatively, co-receptor function may regulate NgR valency at the same site as does agonist binding. Further structural and biochemical studies will be required to define the full implications of the fact that Lingo-1 binding sites on NgR are similar to ligand binding sites.

TABLE II
Summary of NgR mutants: list of residues mutated to alanine

| No binding | Binding to all ligands | Differential binding |
|---|---|---|
| 163 | 61 | 82 |
| 133, 136 | 92 | 108 |
| 158, 160 | 122 | 139 |
| 182, 186 | 127 | 210 |
| 232, 234 | 131 | 78, 81 |
| 82, 179 | 138 | 87, 89 |
| 67, 68, 71 | 151 | 89, 90 |
| 111, 113, 114 | 176 | 95, 97 |
| 114, 117, 163 | 179 | 108, 131 |
| 182, 186, 210 | 227 | 256, 259 |
| 210, 232, 234 | 250 | 36, 38, 61 |
| 67, 68, 95, 97 | D259N | 95, 97, 122 |
| 87, 89, 133, 136 | 36, 38 | 114, 117, 139 |
| 182, 186, 158, 160 | 63, 65 | 117, 119, 120 |
| 111, 113, 114, 138 | 114, 117 | 216, 218, 220 |
| 117, 119, 120, 139 | 127, 151 | 220, 223, 224 |
| 202, 205, 227, 250, | 127, 176 | 237, 256, 259 |
| 277, 279 | | |
| 95, 97, 188, 189, | 143, 144 | 256, 259, 284 |
| 191, 192 | | |
| 95, 97, 117, 119, | 189, 191 | 61, 108, 131 |
| 120, 188, 189 | | |
| | 196, 199 | 63, 65, 87, 89 |
| | 202, 205 | 237, 256, 284 |
| | 267, 269 | 196, 199, 220, 223, 224 |
| | 277, 279 | 211, 213, 237, 256, 259, |
| | | 284 |
| | 189, 191, 237 | 189, 191, 211, 213, 237, |
| | | 256, 259, 284 |
| | 189, 191, 284 | |
| | 202, 205, 227 | |
| | 202, 205, 250 | |
| | 296, 297, 300 | |
| | 171, 172, 175, 176 | |
| | 292, 296, 297, 300 | |
| | 171, 172, 175, 176, | |
| | 196, 199 | |

Binding of Alanine substituted NgR mutants to NgR ligands were compared to wild type NgR and the levels of binding were categorized as ++ (WT level), + (weaker than wild type), tr (trace binding), − (no binding), N/A (not determined). NgR mutant proteins were also subjected to SDS-PAGE and probed by anti-NgR antibodies. Mutants with expression level similar to WT NgR were labeled as “y”.

TABLE III
List of NgR mutants that show differential binding to NgR ligands

| | | | | | | | | NgR |
|---|---|---|---|---|---|---|---|---|
| | | | | | | | | band |
| Resi- | | | B4C- | | | | anti- | West- |
| dues | Ng66 | B4C | 66 | Lingo-1 | OMgp | MAG | NgR | ern |
| WT | ++ | ++ | ++ | ++ | ++ | ++ | ++ | y |
| 82 | ++ | ++ | ++ | + | + | − | ++ | y |
| 108 | ++ | ++ | ++ | tr | + | − | ++ | y |
| 139 | + | ++ | ++ | − | tr | − | ++ | y |
| 210 | + | + | + | − | − | − | + | y |
| 108, 131 | + | + | + | − | + | tr | ++ | y |
| 256, 259 | ++ | ++ | ++ | ++ | + | − | ++ | y |
| 78, 81 | ++ | ++ | ++ | tr | ++ | N/A | ++ | y |
| 87, 89 | ++ | ++ | ++ | − | + | + | ++ | y |
| 89, 90 | + | + | + | − | − | − | ++ | y |
| 95, 97 | + | ++ | + | + | tr | tr | ++ | y |
| 95, 97, | + | + | + | − | tr | tr | ++ | y |
| 122 | | | | | | | | |
| 36, 38, 61 | + | ++ | ++ | tr | + | tr | ++ | y |
| 114, 117, | + | + | + | − | − | − | ++ | y |
| 139 | | | | | | | | |
| 117, 119, | ++ | ++ | ++ | tr | tr | − | ++ | y |
| 120 | | | | | | | | |
| 216, 218, | ++ | ++ | ++ | + | + | tr | ++ | y |
| 220 | | | | | | | | |
| 220, 223, | + | + | + | − | − | tr | ++ | y |
| 224 | | | | | | | | |
| 237, 256, | tr | tr | + | + | tr | − | ++ | y |
| 259 | | | | | | | | |
| 256, 259, | + | + | ++ | ++ | + | − | ++ | y |
| 284 | | | | | | | | |
| 61, 108, | tr | + | + | − | − | − | ++ | y |
| 131 | | | | | | | | |
| 63, 65, 87, | ++ | ++ | ++ | − | + | − | ++ | y |
| 89 | | | | | | | | |
| 237, 256, | ++ | ++ | ++ | ++ | + | − | ++ | y |
| 284 | | | | | | | | |
| 211, 213, | − | − | − | ++ | − | − | ++ | y |
| 237, 256, | | | | | | | | |
| 259, 284 | | | | | | | | |
| 189, 191, | − | − | − | ++ | − | N/A | ++ | y |
| 211, 213, | | | | | | | | |
| 237, 256, | | | | | | | | |
| 259, 284 | | | | | | | | |

Alanine substituted NgR mutants were tested for their binding to AP-Nogo66, AP-B4C, AP-B4C66, AP-Lingo-1, AP-OMgp and AP-MAG and they fall into three categories: (1) Mutants that lose binding to all NgR ligands. (2) Mutants that still maintain binding to all NgR ligands. (3) Differential binding mutants that still bind some ligands but lose binding to other ligands. The D259N mutant is an asparagine substitution to mimic a human polymorphism.

### Example 5

### Juxtaposition of Two NgR Binding Domains from Nogo-A Creates High Affinity Agonist Activity

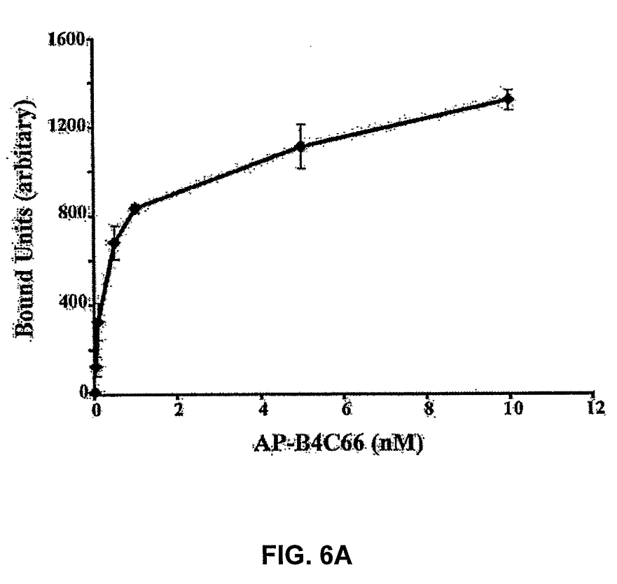

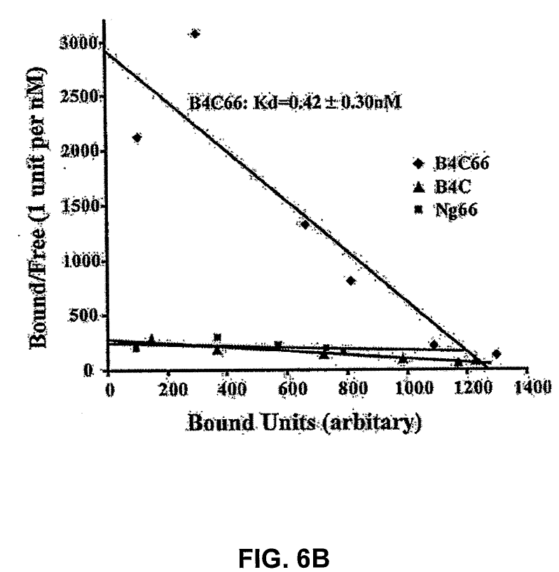

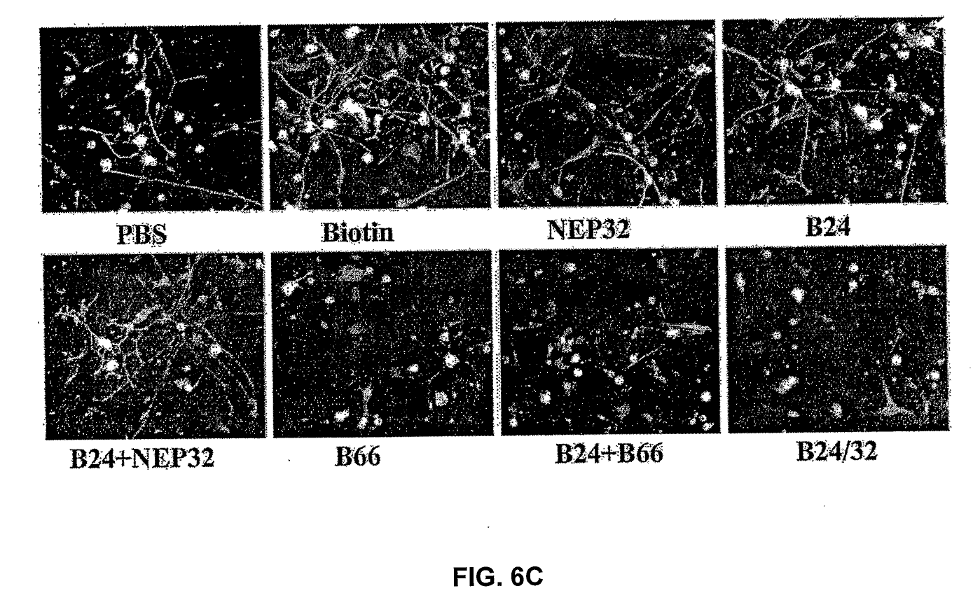

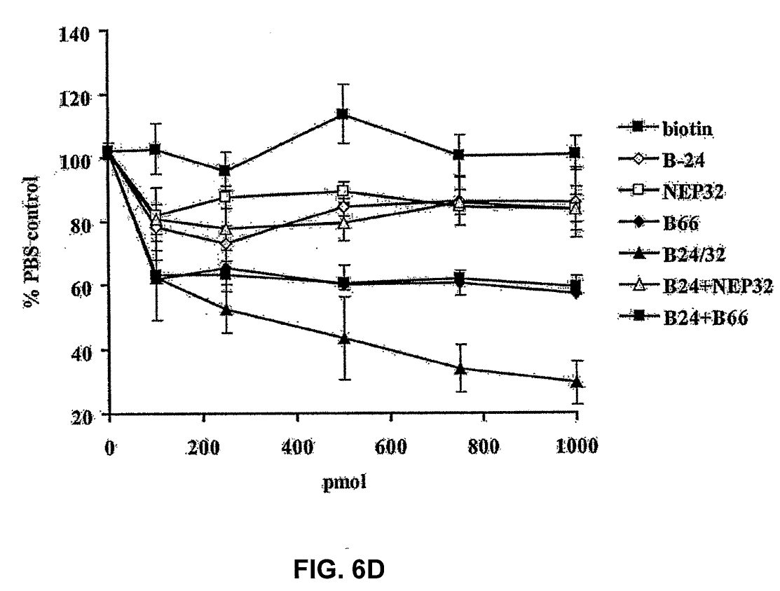

We considered whether the Nogo-66 and Amino-Nogo domains can bind simultaneously to NgR. If the two domains bind simultaneously to receptor, a fusion of the two domains may possess an enhanced receptor affinity based on two-site binding. For intact Nogo-A these two domains may be adjacent to one another at the plasma membrane surface, since they are separated in the primary structure by a hydrophobic loop that extends into the lipid bilayer (Oertle, T., et al., J. Neurosci. 23:5393-5406 (2003b)). In order to create a soluble, tagged ligand resembling this conformation, we generated an AP fusion protein with the B4C fragment of Amino-Nogo-A fused directly to Nogo-66 as described above. The affinity of this AP-B4C-66 ligand for NgR is substantially greater than is that of AP-B4C or AP-Nogo-66. The Kd for this binding is subnanomolar (FIGS. 6A and 6B). Thus, bivalent binding of two linked Nogo-A domains creates a significantly more potent NgR ligand.

Amino-Nogo-A-19 binding does not activate NgR to inhibit axonal outgrowth. However, fusion of this domain to Nogo-66 creates a bivalent ligand for NgR with substantially enhanced receptor affinity. While not being bound by theory, this enhanced affinity may explain the finding that in vitro and in vivo assays indicate a greater role for Nogo-A than MAG in limiting axonal growth, despite the greater abundance of MAG protein in myelin preparations.

Next we considered the effect of these two peptide domains on neurite outgrowth. While a synthetic Nogo-66 peptide fragment inhibits neurite outgrowth by binding to NgR as an agonist, shorter Nogo-66 peptides bind to NgR as antagonists and do not alter outgrowth. Previously, we demonstrated the antagonistic activity of a peptide composed of the amino terminal 40 aa of Nogo-66 (NEP1-40) (GrandPre, T., et al., Nature 417:547-551 (2002)). Similar NgR antagonistic results are obtained for peptides as short as 32 aa (data not shown), suggesting that the 33-66 region is required for receptor activation but not high affinity binding (GrandPre, T., et al., Nature 417:547-551 (2002)). Shorter fragments of Nogo-66 do not interact with NgR (GrandPre, T., et al., Nature 417:547-551 (2002) and data not shown). The carboxyl 24 aa segment of Amino-Nogo-A mediates AP fusion protein binding to NgR (FIGS. 1C and 1D) but this peptide does not block or enhance Nogo-66 action on neurite outgrowth (FIGS. 6C and 6D).

We reasoned that fusing the 24 aa segment of Amino-Nogo-A to NEP32 antagonist peptide might create a high affinity antagonist with a potency similar to the binding of AP-B4C-66 to NgR. To examine this hypothesis, a biotinylated peptide containing the Amino-Nogo-24 sequence fused at its carboxyl terminus to NEP32 was synthesized. Biotin labeled Ng24 (biotin-IFSAELSKTSVVDLLYWRDIKKTG) and 24/32 (B24/32: biotin-IFSAELSKTSVVDLLYWRDIKKTGGRIYKGVIQAIQKSDEGHP FRAYLESEVAISEE) were synthesized and purified by the W.M. Keck facility at Yale University. For the cDRG outgrowth assay, dissociated E13 cDRG neurons were plated for 6 hours before fixation. Neurons were stained with anti-Neurofilament (Sigma Catalog #N4142) and anti-HuC/D (Molecular Probes A-21271) antibodies. Cell area, number of attached cells and neurite length were measured using the linageexpress machine and software (Axon Instrument).

Unexpectedly, the 24-32 fusion peptide potently inhibited axon outgrowth from DRG neurons (FIGS. 6C and 6D). It is clear that the Amino-Nogo-24 domain can bind to NgR independently but when fused to the NEP32 creates a high affinity Nogo-A selective NgR agonist. Thus, the Nogo-66 (33-66) region is not essential for receptor activation. Instead, the results suggest that bivalent interaction of ligands with NgR may be critical. Since NgR can bind to itself and is clustered in lipid rafts (Fournier, A. E., et al., J. Neurosci. 22:8876-8883 (2002); Liu, B. P., et al., Science 297:1190-1193 (2002)), bivalent ligands may activate receptor through modulation of its aggregation state in the plane of the bilayer.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

1. An isolated polypeptide fragment of 30 residues or less, comprising an amino acid sequence that is at least 90% identical to a reference amino acid sequence selected from the group consisting of
(a) amino acids 995 to 1013 of SEQ ID NO:2;
(b) amino acids 995 to 1014 of SEQ ID NO:2;
(c) amino acids 995 to 1015 of SEQ ID NO:2;
(d) amino acids 995 to 1016 of SEQ ID NO:2;
(e) amino acids 995 to 1017 of SEQ ID NO:2;
(f) amino acids 995 to 1018 of SEQ ID NO:2;
(g) amino acids 992 to 1018 of SEQ ID NO:2;
(h) amino acids 993 to 1018 of SEQ ID NO:2; and
(i) amino acids 994 to 1018 of SEQ ID NO:2;
wherein said polypeptide binds NgR1.

2. The polypeptide fragment of claim 1, wherein said amino acid sequence is at least 95% identical to said reference amino acid sequence.

3. The polypeptide fragment of claim 2, wherein said reference amino acid sequence is identical to said reference amino acid sequence.

4. An isolated polypeptide fragment of 200 residues or less, comprising a first amino acid sequence that is at least 90% identical to amino acids 995 to 1018 of SEQ ID NO:2, where said first amino acid sequence is linked to amino acids 1055 to 1086 of SEQ ID NO:2, and wherein said polypeptide fragment binds NgR1.

5. The polypeptide fragment of claim 4, wherein said first amino acid sequence comprises amino acids 995 to 1018 of SEQ ID NO:2.

6. The polypeptide fragment of claim 5, wherein said first amino acid sequence comprises amino acids 950 to 1018 of SEQ ID NO:2.

7. The polypeptide fragment of claim 4, wherein said polypeptide fragment enhances NgR-mediated neurite outgrowth inhibition.

8. The polypeptide fragment of claim 5, comprising SEQ ID NO:5.

9. The polypeptide fragment of claim 5, consisting essentially of SEQ ID NO:5.

10. The polypeptide fragment of claim 4, wherein said polypeptide fragment is modified.

11. The polypeptide fragment of claim 10, wherein said modification is biotinylation.

12. The polypeptide fragment of claim 1, wherein said polypeptide fragment is linked to a heterologous polypeptide.

13. The polypeptide fragment of claim 12, wherein said heterologous polypeptide is selected from the group consisting of Glutathione S-transferase (GST), histidine tag (His tag), alkaline phosphatase (AP), and Fc.

14. An isolated human NGR1 polypeptide comprising amino acids 27 to 473 of SEQ ID NO:4, except for amino acid substitution at least the amino acid positions selected from the group consisting of:
(a) amino acids 67, 68 and 71;
(b) amino acids 111, 113 and 114;
(c) amino acids 133 and 136;
(d) amino acids 158, 160, 182, and 186;
(e) amino acid 163; and
(f) amino acids 232 and 234;
wherein said NgR1 polypeptide does not bind to any of Nogo 66, OMgp, Mag or Lingo-1.

15. An isolated human NGR1 polypeptide comprising amino acids 27 to 473 of SEQ ID NO:4, except for amino acid substitutions at least the amino acid positions selected from the group consisting of:
(a) amino acids 78 and 81;
(b) amino acids 87 and 89;
(c) amino acids 89 and 90;
(d) amino acids 95 and 97;
(e) amino acid 108;
(f) amino acids 117, 119 and 120;
(g) amino acid 139;
(h) amino acid 210; and
(i) amino acids 256 and 259;
wherein said NgR polypeptide selectively binds to at least one but not all of Nogo 66, OMgp, Mag or Lingo-1.

16. A host cell comprising the polypeptide of claim 14.

17. A composition comprising the polypeptide of claim 1, and a pharmaceutically acceptable carrier.

18. The composition of claim 17, wherein said composition is formulated for administration by a route selected from the group consisting of parenteral administration, subcutaneous administration, intravenous administration, intramuscular administration, intraperitoneal administration, transdermal administration, buccal administration, oral administration and microinfusion administration.

19. The composition of claim 18, wherein said composition further comprises a carrier.
